(19) United States
(12) Patent Application Publication
(10) Pub. No.: US 2015/0202211 A1
(43) Pub. Date: Jul. 23, 2015

(54) PROGESTERONE FORMULATIONS HAVING A DESIRABLE PK PROFILE

(71) Applicant: TherapeuticsMD, Inc., Boca Raton, FL (US)

(72) Inventors: Julia Amadio, Boca Raton, FL (US); Brian Bernick, Boca Raton, FL (US)

(73) Assignee: TherapeuticsMD, Inc., Boca Raton, FL (US)

(21) Appl. No.: 14/671,651

(22) Filed: Mar. 27, 2015

Related U.S. Application Data
(63) Continuation-in-part of application No. 14/125,547, filed on Dec. 11, 2013.
Application No. 14/125,547 is a continuation of application No. PCT/US2013/023309, filed on Jan. 25, 2013.
Application No. PCT/US2013/023309 is a continuation of application No. 13/843,362, filed on Mar. 15, 2013.
Application No. 13/843,362 is a continuation-in-part of application No. 13/843,428, filed on Mar. 15, 2013.
(60) Provisional application No. 61/661,302, filed on Jun. 18, 2012.
(60) Provisional application No. 61/662,265, filed on Jun. 20, 2012.
(60) Provisional application No. 61/972,068, filed on Mar. 28, 2014.

(51) Int. Cl. A61K 31/57

(52) U.S. Cl. CPC A61K 31/57

(57) ABSTRACT

This disclosure provides progesterone formulations, methods of using these formulations, and their related pharmacokinetic parameters. In particular embodiments, the formulations disclosed herein allow for a reduction in the amount of progesterone administered to a patient in need thereof, while still providing the benefits of a larger dosage amount.

### FIELD OF INVENTION

This disclosure relates to progesterone formulations, methods of using these formulations, and their related pharmacokinetic parameters. Various progesterone formulations may be used in hormone therapies for menopausal, peri-menopausal and post-menopausal females, for example, to mitigate side effects from estrogen replacement therapy. In addition, various progesterone formulations may be used to prevent preterm delivery in pregnant women having a shortened cervix. Progesterone can likewise be used to treat endometrial hyperplasia and amenorrhea.

### BACKGROUND OF THE INVENTION

It is not uncommon for pre-menopausal, peri-menopausal, menopausal, or postmenopausal females, to experience vaginal dryness, vaginal odor, vulvar irritation and itching, dysuria (pain, burning or stinging when urinating), dysparuenia (vaginal pain associated with sexual activity), or vaginal bleeding associated with sexual activity. They may also experience night sweats and menopausal hot flashes (vasomotor symptoms), soreness, increased or variant urinary frequency and urgency, urinary discomfort and incontinence (“estrogen-deficient urinary state(s)”), mood disturbances, and symptoms related vulvo-vaginal atrophy, endometrial hyperplasia, endometrial cancer, and other symptoms of estrogen-related disorders. These symptoms, and other symptoms known to those skilled in the art, are believed to be induced as a result of inadequate or irregular hormone production. As a result, prophylactic methods and treatment regimens to alleviate these symptoms frequently include low dosages of estrogens.

But increased levels of estrogens, including estradiol, whether due to prescription or naturally-occurring increases, may lead to the symptoms and disorders previously mentioned. To mitigate the effect of increased estradiol levels on the endometrium, progesterone administration is often a prophylactic method or prescribed treatment to prevent the negative effects of estrogens such as endometrial hyperplasias and related disorders.

These prophylactic methods and prescribed treatments involving the use of one or more of a group of medications designed to supplement hormone levels in women who experience irregular or decreased hormone production or who lack adequate hormone production, may generally be referred to as hormone replacement therapy (HRT).

Hormone replacement therapy (HRT) is a medical treatment that involves the use of one or more of a group of medications designed to supplement hormone levels in women who lack adequate hormone production. It can mitigate and prevent symptoms caused by diminished circulating estrogen and progesterone hormones.

HRT is available in various forms. One therapy involves administration of low dosages of one or more estrogen(s) or one or more chemical analogues. Another involves administration of progesterone or one or more chemical analogues. Among other effects, progesterone administration acts to mitigate certain undesirable side effects from estradiol administration or naturally-occurring elevated blood levels including endometrial hyperplasia (thickening) and prevention or inhibition of endometrial cancer. Progesterone is a C-21 steroidal sex hormone involved in the female menstrual cycle, pregnancy (supports gestation) and embryogenesis of humans and other species. Progesterone belongs to a class of hormones called progestogens, and is the major naturally occurring human progestogen. Like other steroids, progesterone consists of four interconnected cyclic hydrocarbons. Progesterone is hydrophobic, having a reported aqueous solubility of 0.007±0.0 mg/ml. Progesterone is poorly absorbed when administered orally.

Existing progesterone prophylactic methods and prescribed treatments inconsistently or irregularly achieve high levels of absorbed progesterone at low dosages of progesterone. Existing methods and treatments often use synthetic progestins. Synthetic progestins such as medroxyprogesterone acetate or norethindrone acetate have been specifically designed to resist enzymatic degradation and remain active after oral administration. However, these compounds exert undesirable effects on the liver (notably on lipids) and often cause psychological side effects that can be severe enough to contraindicate their use.

One conventional progesterone therapeutic is PROMETRIUM (progesterone, USP) (Abbott Laboratories, Chicago, Ill.). PROMETRIUM is an FDA-approved drug, formulated in a peanut oil-based medium, containing micronized progesterone, but with a relatively large particle size fraction. The active ingredient in PROMETRIUM is considered to be structurally identical to naturally occurring progesterone produced by a woman's body (also known as a “bioidentical”).

Clinical trials involving PROMETRIUM have shown significant intra- and inter-patient variability. For example, a clinical trial involving postmenopausal women who were administered PROMETRIUM once a day for five days resulted in the mean pharmacokinetic parameters listed in Table 1 (see Table 1, package insert for PROMETRIUM).

TABLE 1
Pharmacokinetic Parameters of PROMETRIUM Capsules

PROMETRIUM Capsules Daily Dose

| Parameter | 100 mg | 200 mg | 300 mg |
|---|---|---|---|
| Cₘₐₓ (ng/ml) | 17.3 ± 21.9 | 38.1 ± 37.8 | 60.6 ± 72.5 |
| Tₘₐₓ (hr) | 1.5 ± 0.8 | 2.3 ± 1.4 | 1.7 ± 0.6 |
| AUC (0-10) | 43.3 ± 30.8 | 101.2 ± 66.0 | 175.7 ± 170.3 |
| (ng × hr/ml) | | | |

The unusually high variability in Cₘₐₓ and AUC, as evidenced by the large reported standard deviation, may indicate that a significant percentage of patients are overdosed or receive a sub-optimal dose.

The presence of peanut oil in the formulation excludes patients who are allergic to peanut oil. Peanut oil, like other peanut products, may act as an allergen. Indeed, there is a portion of the population that has severe reactions to peanut oil. Peanut allergies are becoming a significant health concern. Food allergies are a leading cause of anaphylaxis, with approximately 200 deaths occurring annually in the United States. While incidence and prevalence are not entirely known, it is suspected that about 6% of children and 4% of adults in North America are affected by food allergies. Many food allergies experienced by children are generally outgrown in adulthood with the exception of peanut allergies.

Progesterone and its analogues can be used to treat a variety of medical conditions, including acute diseases or disorders, as well as chronic diseases and disorders associated with long-term declines of natural progesterone levels.

Accordingly, improved formulations of progesterone would be advantageous. To that end, and disclosed herein, are, among other things, a new softgel progesterone pharmaceutical composition containing solubilized or partially solubilized progesterone, suspended progesterone, a solubilizing agent, and a non-ionic surfactant.

### SUMMARY OF THE INVENTION

Various pharmaceutical formulations are disclosed herein. For example, pharmaceutical formulations are disclosed comprising ultra-micronized progesterone. Moreover, pharmaceutical formulations are disclosed comprising formulations of ultra-micronized progesterone, wherein the ultra-micronized progesterone is combined with a suitable excipient.

Thus, in various illustrative embodiments, the invention comprises an encapsulated liquid pharmaceutical formulation for orally administering progesterone to a mammal in need thereof, said formulation comprising: progesterone, as the sole active pharmaceutical ingredient. The progesterone can be fully solubilized, or, more typically, partially solubilized, in a solubilizing agent, with any insoluble progesterone being suspended in the solubilizing agent. The solubilizing agent can comprise a medium chain fatty acid-polyolester or a mixture of medium chain fatty acid-polyol esters. The polyol can be, for example, a glycol such as ethylene glycol, polyethylene glycol, propylene glycol, polypropylene glycol, etc. In other embodiments, the polyol can be a triol such as glycerol. When the polyol is a glycol, the glycol can be mono- or di-esterified with a given fatty acid (simple) or can be a mixed di-ester using different medium chain fatty acids. When the polyol is glycerol, the glycerol can be mono-, di-, or tri-esterified giving a monoglyceride, diglyceride, or triglyceride. Typical di- and triglycerides are simple triglycerides, though in certain embodiments, the di- and triglycerides can be mixed. In particular, embodiments, the solubilizing agent can comprise a simple, mixed, or combination simple and mixed glycol di-ester. In still other embodiments, the solubilizing agent can be a simple, mixed, or combination simple and mixed triglyceride. For example, in a particular embodiment, the solubilizing agent can comprise an oil having simple and mixed triglycerides prepared from predominantly C8 and C10 fatty acids. An example of such a triglyceride is MIGLYOL® 812.

In certain embodiments, the formulation can further comprise a non-ionic surfactant. As discussed elsewhere herein, the non-ionic surfactant can comprise GELUCIRE 44/14.

In certain embodiments the progesterone is micronized or ultra-micronized. In certain embodiments, at least about 80 wt % of the total progesterone is micronized. The fatty acids can be predominantly (>50 wt %): C6 to C12 fatty acids, C6 to C10 fatty acids, C8 to C12 fatty acids, or C8 to C10 fatty acids. Some embodiments comprise a non-ionic surfactant that comprises C8 to C18 fatty acid esters of glycerol and polyethylene glycol.

In other embodiments, a softgel progesterone pharmaceutical composition as a hormone replacement therapy (HRT), or as a prophylactic method or a prescribed treatment to mitigate the associated symptoms associated with irregular or inadequate hormone levels is provided.

In certain embodiments, this disclosure provides a pharmaceutical composition for administering progesterone to subject in need thereof, the composition comprising an amount of progesterone and one or more solubilizing agents, wherein upon administration to the subject in need thereof, the composition produces at least one the following pharmacokinetic parameters in said subject an AUC₀₋ₜ in (ng/ml)*hr of from about 5 to about 500; an AUC_{0-∞} in (ng/ml)*hr of from about 5 to about 500; or a Cₘₐₓ in ng/ml of from about 3 to about 350; wherein the amount of progesterone is less than 200 mg.

In certain embodiments, the AUC₀₋ₜ is from about 5 to about 400, from about 5 to about 300, from about 5 to about 240, from about 20 to about 200, from about 25 to about 150, or from about 25 to about 140.

In still other embodiments, AUC₀₋ₜ is 120 (ng/ml)*hr±95%.

In some embodiments, the AUC_{0-∞} is from about 5 to about 400, from about 5 to about 300, from about 5 to about 270, from about 20 to about 200, from about 25 to about 150, or from about 25 to about 140.

In certain other embodiments, the AUC_{0-∞} is 137 (ng/ml)*hr±95%.

In certain embodiments, the Cₘₐₓ in ng/ml is from about 3 to about 325, from about 3 to about 300, from about 3 to about 250, from about 3 to about 240, or from about 3 to about 230.

In other embodiments, the Cₘₐₓ is 75 ng/ml±95%.

In certain embodiments, the amount of progesterone in the composition is about 150 mg.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure.

FIG. 1 illustrates a process to produce fill material in accordance with various embodiments;

FIG. 2 illustrates a process to produce softgel capsules in accordance with various embodiments;

FIG. 3 illustrates a process to produce softgel capsules in accordance with various embodiments; and

FIG. 4 illustrates a dissolution study of a formulation in accordance with various embodiments.

FIG. 5 illustrates a graph of the particle distribution obtained in Example 10.

FIG. 6 illustrates a dissolution study of a formulation in accordance with various embodiments of the invention.

FIG. 7 illustrates a Linear Plot of Mean Plasma Progesterone (Corrected) Concentrations Versus Time (N=62).

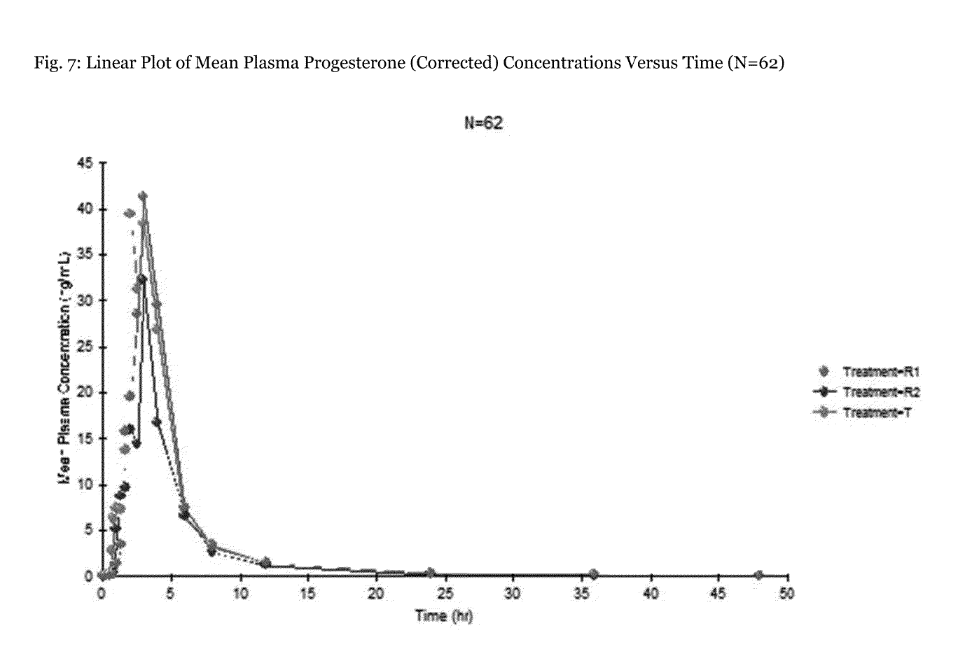

FIG. 8 illustrates a graph that is a Semi-logarithmic Plot of Mean Plasma Progesterone (Corrected) Concentrations Versus Time (N=62)

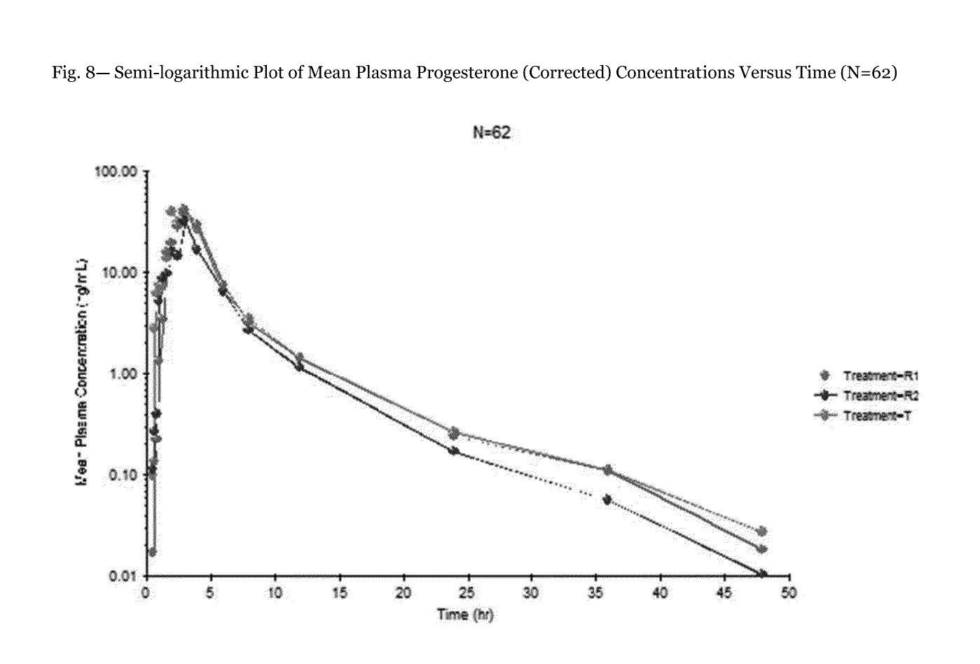

FIG. 9 illustrates a graph that is a Linear Plot of Mean Plasma Progesterone (Uncorrected) Concentrations Versus Time (N=62)

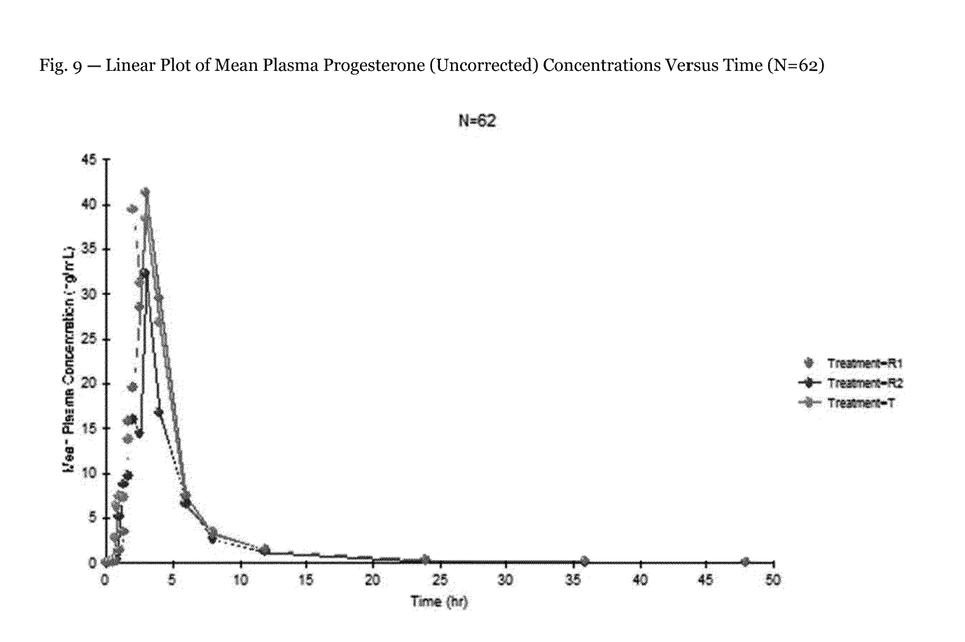

FIG. 10 illustrates a graph that is a Semi-logarithmic Plot of Mean Plasma Progesterone (Uncorrected) Concentrations Versus Time (N=62)

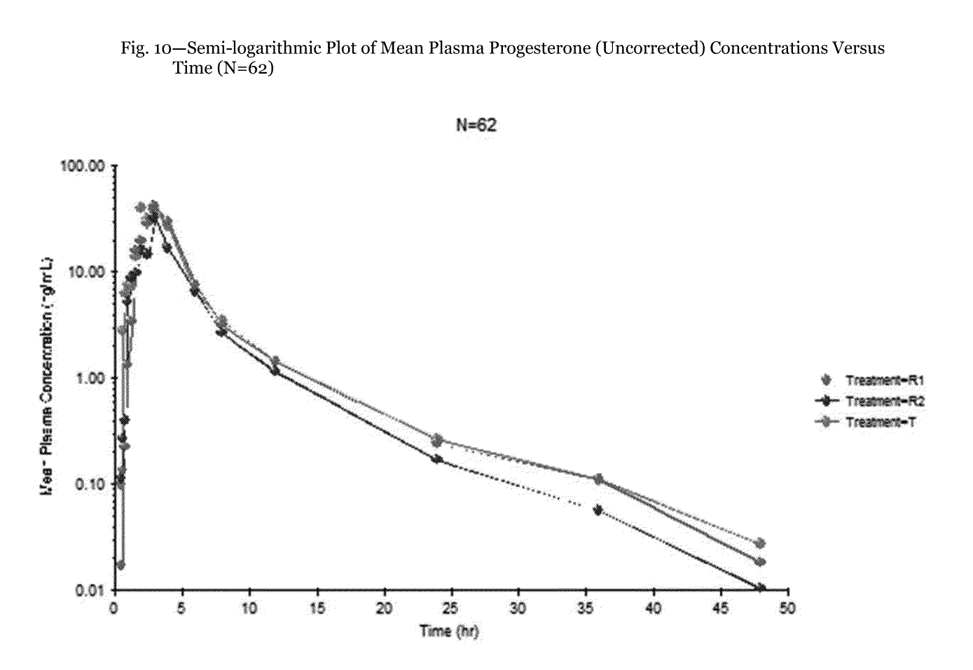

### DETAILED DESCRIPTION

This disclosure provides a pharmaceutical formulation comprising progesterone and a solubilizing agent. In some embodiments, a pharmaceutical formulation comprising ultra-micronized progesterone is provided. As described in detail herein, various solubilizing agents, lubricants, and other excipients may be included. In further embodiments, ultra-micronized progesterone formulations provide improved bioavailability and other pharmacokinetic improvements. These embodiments are described in sufficient detail to enable those skilled in the art to practice these embodiments. Further, other embodiments may be used and other changes may be made without departing from the scope of this disclosure. The following detailed description is therefore not to be taken in a limiting sense. As used in this disclosure, the term “or” is a logical disjunction and does not indicate an exclusive disjunction unless expressly indicated as such with the terms “either,” “unless,” “alternatively,” and words of similar effect.

### DEFINITIONS

Unless otherwise specified, the following definitions apply.

The phrase “active pharmaceutical ingredient” or “API” as used herein, means the active compound(s) used in formulating a drug product. In exemplary embodiments, the API is progesterone.

The term “bioequivalent” has the meaning prescribed in 21 CFR §320.1(e), e.g. the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the Cₘₐₓ, AUC, or, optionally, Tₘₐₓ is within 80.00% to 125.00%.

The term “bioidentical” or “natural” used in conjunction with the hormones disclosed herein, means hormones that are identical to or match the chemical structure and effect of those that occur naturally or endogenously in the human body. An exemplary natural estrogen is estradiol.

The term “drug product” as used herein means at least one API in combination with at least one excipient, wherein the API and at least one excipient are provided in unit dosage form.

The term “estrogen” means generally the different hormone types of estrogen, synthetically or naturally occurring, including estradiol, estriol, and estrone.

The term “estradiol” means (17β)-estra-1,3,5(10)-triene-3,17-diol. Estradiol is also called 17β-estradiol, oestradiol, or E2 and is found endogenously in the human body. Irrespective of the what it is called, estradiol refers to the bio-identical form of estradiol found in the human body having the structure:

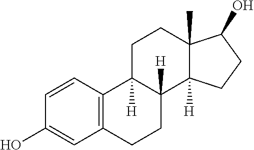

Estradiol is supplied in an anhydrous or a hemi-hydrate form; for the purposes of this disclosure, the anhydrous form or the hemihydrate form can be substituted for the other by accounting for the water or lack of water according to well-known and understood techniques.

The phrase “equivalent dosage form” as used herein refers to a dosage form that is identical to a reference dosage form in composition (e.g. identical solubilizing agent(s), non-ionic surfactant(s), and API), but differs from the reference dosage form in the amount of API present or in the ratio of the various components in the reference dosage form.

The term “ultra-micronized progesterone,” as used herein, refers to micronized progesterone having an X50 particle size value below about 20 microns or having an X90 value below about 25 microns. The term “X50” as used herein, means that half of the particles in a sample are smaller in diameter than a given number. For example, ultra-micronized progesterone having an X50 of 5 microns means that, for a given sample of ultra-micronized progesterone, half of the particles have a diameter of less than 5 microns. In that regard, similar terms, in the form XYY mean that YY percent of the particles in the sample are smaller in diameter than a given number. For example, X90 means that ninety percent of the particles in a sample are smaller in diameter than a given number.

The term “administer,” “administration,” “deliver” or “delivery” (collectively “administration”), as used herein, means oral administration of the formulation disclosed herein, preferably in a soft gelatin capsule.

The term “glyceride” is an ester of glycerol (1,2,3-propanetriol) with acyl radicals of fatty acids and is also known as an acylglycerol. If only one position of the glycerol molecule is esterified with a fatty acid, a “monoglyceride” is produced; if two positions are esterified, a “diglyceride” is produced; and if all three positions of the glycerol are esterified with fatty acids, a “triglyceride” or “triacylglycerol” is produced. A glyceride is “simple” if all esterified positions contain the same fatty acid; whereas a glyceride is “mixed” if the esterified positions contained different fatty acids. The carbons of the glycerol backbone are designated sn-1, sn-2 and sn-3, with sn-2 being in the middle carbon and sn-1 and sn-3 being the end carbons of the glycerol backbone.

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain 6 (C6) to 14 (C4) carbon atoms, 8 (C8) to 12 (C12) carbon atoms, or 8 (C8) to 10 (C10) carbon atoms.

The terms “medium chain fatty acid” and “medium chain fatty acid derivative” are used to describe fatty acids or fatty acid derivatives with aliphatic tails (i.e., carbon chains) having 6 to 14 carbon atoms. Fatty acids consist of an unbranched or branched aliphatic tail attached to a carboxylic acid functional group. Fatty acid derivatives include, for example, fatty acid esters and fatty acid containing molecules, including, without limitation, mono-, di- and triglycerides that include components derived from fatty acids. Fatty acid derivatives also include fatty acid esters of ethylene or propylene glycol. The aliphatic tails can be saturated or unsaturated (one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. Examples of medium chain fatty acids include, without limitation, caproic acid, caprylic acid, capric acid, lauric acid, myristic acid, and derivatives thereof.

The term “oil,” as used herein, refers to any pharmaceutically acceptable oil, especially medium chain oils, and specifically excluding peanut oil, that can suspend and/or solubilize bioidentical progesterone and/or estradiol, including starting materials and/or precursors thereof, including micronized progesterone and/or micronized estradiol as described herein.

The term “medium chain oil” refers to an oil wherein the composition of the fatty acid fraction of the oil is predominantly medium chain (i.e., C6 to C14) fatty acids, i.e., the composition profile of fatty acids in the oil is predominantly medium chain. As used herein, “predominantly” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. Those of skill in the art that will readily appreciate that the terms “alkyl content” or “alkyl distribution” of an oil can be used in place of the term “fatty acid fraction” of an oil in characterizing a given oil or solubilizing agent, and these terms are used interchangeable herein. As such, medium chain oils suitable for use in the formulations disclosed herein include medium chain oils wherein the fatty acid fraction of the oil is predominantly medium chain fatty acids, or medium chain oils wherein the alkyl content or alkyl distribution of the oil is substantially medium chain alkyls (C6-C12 alkyls). It will be understood by those of skill in the art that the medium chain oils suitable for use in the formulations disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3×16=48. Conversely, a triglyceride with an ECN=40 may have “mixed” fatty acid chain lengths of 8, 16 and 16; 10, 14 and 16; 8, 14 and 18; etc. Naturally occurring oils are frequently “mixed” with respect to specific fatty acids, but tend not to contain both long chain fatty acids and medium chain fatty acids in the same glycerol backbone. Thus, triglycerides with ECN's of 21-42 typically contain predominately medium chain fatty acids; while triglycerides with ECN's of greater than 43 typically contain predominantly long chain fatty acids. For example, the ECN of corn oil triglyceride in the USP would be in the range of 51-54. Medium chain diglycerides with ECN's of 12-28 will often contain predominately medium chain fatty chains, while diglycerides with ECN's of 32 or greater will typically contain predominately long chain fatty acid tails. Monoglycerides will have an ECN that matches the chain length of its sole fatty acid chain. Thus, monoglyceride ECN's in the range of 6-14 contain mainly medium chain fatty acids, and monoglycerides with ECN's 16 or greater will contain mainly long chain fatty acids.

The average ECN of a medium chain triglyceride oil is typically 21-42. For example, as listed in the US Pharmacopeia (USP), medium chain triglycerides having the following composition as the exemplary oil in the table below

| Fatty-acid Tail Length | % of oil | Exemplary Oil |
|---|---|---|
| 6 | ≦2.0 | 2.0 |
| 8 | 50.0-80.0 | 70.0 |
| 10 | 20.0-50.0 | 25.0 |
| 12 | ≦3.0 | 2.0 |
| 14 | ≦1.0 | 1.0 |

would have an average ECN of 3*[(6*0.02)+(8*0.70)+(10*0.25)+(12*0.02)+(14*0.01)]=25.8. The ECN of the exemplary medium chain triglycerides oil can also be expressed as a range (per the ranges set forth in the USP) of 24.9-27.0. For oils that have mixed mono-, di-, and trigylcerides, or single and double fatty acid glycols, the ECN of the entire oil can be determined by calculating the ECN of each individual component (e.g., C8 monoglycerics, C8 diglycerides, C10 monoglycerides, and C10 monoglycerides) and taking the sum of the relative percentage of the component multiplied by the ECN normalized to a monoglyceride for each component. For example, the oil having C8 and C10 mono- and diglycerides shown in the table below has an ECN of 8.3, and is thus a medium chain oil.

| | | ECN as % of oil | ECN as % of |
|---|---|---|---|
| Fatty-acid | | (chain length) × | oil normalized |
| Tail Length | % of oil | (% in oil) | to monoglyceride |
| C8 monoglyceride | 47 | 8 × 0.47 = 3.76 | 3.76 |
| C10 monoglyceride | 8 | 10 × 0.08 = 0.8 | 0.8 |
| C8 diglyceride | 38 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN | | | 8.3 |
| (normalized to | | | |
| monoglycerides) | | | |

Expressed differently, ECN can be calculated as each chain length in the composition multiplied by its relative percentage in the oil: (8*0.85)+(10*0.15)=8.3.

The term “patient” refers to a human individual who has received, who might receive, or is receiving health or pharmaceutical care, or is under the supervision and care of a physician, pharmacist, or medically trained professional. This individual may be expecting this care, may be currently receiving it, or may have already received it.

The term “progesterone” refers to pregn-4-ene-3,20-dione. Progesterone is also interchangeably called P4 and is found endogenously in the human body. As used herein, progesterone refers to the bio-identical or body-identical form of progesterone found in the human body having the structure:

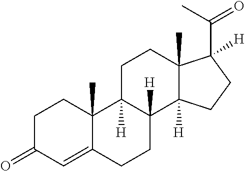

The term “solubilized progesterone” means that the progesterone or a portion thereof is solubilized or dissolved in the solubilizing agent(s) or the formulations disclosed herein. In some embodiments, the progesterone is “partially solubilized” with a portion of the progesterone being solubilized or dissolved in the solubilizing agent and a portion of the progesterone being suspended in the solubilizing agent. Partially solubilized progesterone may include progesterone that is about 1% solubilized, about 5% solubilized, about 10% solubilized, about 15% solubilized, or about 20% solubilized, about 30% solubilized, about 40% solubilized, about 50% solubilized, about 60% solubilized, about 70% solubilized, about 80% solubilized, about 85% solubilized, about 90% solubilized or about 95% solubilized. In other embodiments, the progesterone is “fully solubilized” with all or substantially all of the progesterone being solubilized or dissolved in the solubilizing agent. Fully solubilized progesterone may include progesterone that is about 97% solubilized, about 98% solubilized, about 99% solubilized or about 100% solubilized. In particular embodiments, the progesterone is less than about 20% solubilized. Solubility can be expressed as a mass fraction (% w/w, which is also referred to as wt %).

The term “pharmaceutical composition” refers to a composition comprising at least a solubilizing agent and progesterone. As used herein, pharmaceutical compositions are delivered, for example via oral administration. Furthermore, as used herein, “pharmaceutical composition” and “formulation” are used interchangeably.

The term “uniform distribution” means at least one of uniform dispersion, solubility, or lack of agglomeration of progesterone in gastric juices compared to PROMETRIUM.

The term “gastric juices” means the watery, acidic digestive fluid that is secreted by various glands in the mucous membrane of the stomach and consists chiefly of hydrochloric acid, pepsin, rennin, and mucin.

The term “excipients,” as used herein, refers to non-API substances such as solubilizing agents, anti-oxidants, oils, lubricants and others used in formulating pharmaceutical products. They are generally safe for administering to humans according to established governmental standards, including those promulgated by the United States Food and Drug Administration.

The term “carrier,” as used herein, means any substance or mixture of substances that may be mixed with or contain an API (e.g., ultra-micronized progesterone). The term carrier is interchangeable with solubilizing agent.

The term “capsule,” as used herein, refers to a generally safe, readily dissolvable enclosure for carrying certain pharmaceutical products, and includes hard or soft shell capsules.

The term “softgel,” includes soft shell capsules, including soft-gelatin capsules and soft vegetable-based capsules, and soft capsules made from other materials providing the composition of such soft capsules are compatible with the formulations of the various embodiments described herein. A softgel may comprise two primary phases: a gel or vegetable-based capsule and a fill material of the pharmaceutical formulation as described herein. In particular embodiments, the weight of the fill material does not exceed 500 mg, i.e. the fill material weighs less than 500 mg, less than 450 mg, less than 400 mg, less than 350 mg, less than 300 mg, less than 250 mg, less than 200 mg, or less than 150 mg.

The term “bioavailability” has the meaning prescribed in 21 CFR §320.1(a): the rate and extent to which the active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action. For example, bioavailability can be measured as the amount of API in the blood (serum or plasma) as a function of time. Pharmacokinetic (PK) indicators such as AUC, Cₘₐₓ, or Tₘₐₓ may be used to measure and assess bioavailability. Absorption as used in this definition can include absorption in the stomach, intestines, or other tissue that help facilitate absorption of the API into the bloodstream.

The term “co-administered” as used herein, means that two drug products are administered simultaneously or sequentially on the same or different days.

The terms “pharmacokinetics,” “pharmacokinetic measurements,” “pharmacokinetic parameters,” and “PK parameters” refers to parameters or measures used to assess bioavailability such as AUC, Cₘₐₓ, or Tₘₐₓ include assessments and determinations to study absorption, distribution, metabolism, and excretion of a drug.

The term “reference listed drug product” (“RLD”) means PROMETRIUM (progesterone, USP) (Abbott Laboratories, Chicago. Ill.). PROMETRIUM is an FDA-approved drug, formulated in a peanut oil-based medium, containing micronized progesterone, but with a relatively large particle size fraction.

The term “secretory activity” refers to complete and partial secretory activity of the endometrium as is well understood in the art and as is discussed at length in Noyes. R. W., Hertig, A. T. and Rock, J. (1950), Dating the endometrial biopsy. Fertil. Steril., 1, 3-25, which is incorporated herein by reference. See also, Deliqdisch, L., (1993), Effects of hormone therapy on the endometrium. Mod Pathol. January, vol. 6(1), pp 94-106, which is incorporated herein by reference. Noyes et al., is also referenced for additional information regarding endometrial biopsies.

The term “solubilized” refers to the amount of an API that is in solution. Solubility and percent solubility are expressed herein as a mass fraction (mg/g) or (% w/w, also referred to as wt. %).

The term “solubilizing agent” refers to an agent or combination of agents that solubilize an active pharmaceutical ingredient (e.g., estradiol or progesterone). For example and without limitation, suitable solubilizing agents include medium chain oils and other solvents and co-solvents that solubilize or dissolve an active pharmaceutical ingredient to a desirable extent. Solubilizing agents suitable for use in the formulations disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting formulation. Examples of such excipients include, but are not limited to, surfactants, emulsifiers, thickeners, colorants, flavoring agents, etc. In some embodiments, the solubilizing agent is a medium chain oil and, in some other embodiments, the medium chain oil is combined with a co-solvent(s) or other excipient(s).

The term “subject” refers to both human and non-human animal subjects who are administered the pharmaceutical composition of this disclosure. Specifically intended are mammalian subjects. More specifically intended are human subjects.

The term “area under the curve” or “AUC” refers to the area under the curve defined by changes in the blood concentration of an active pharmaceutical ingredient (e.g., progesterone), or a metabolite of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone), or a metabolite of the active pharmaceutical ingredient, over time.

The term “Tₘₐₓ” refers to the time that it takes for the blood concentration of an active pharmaceutical ingredient (e.g., estradiol or progesterone), or a metabolite of the active pharmaceutical ingredient, to reach the maximum value.

Optionally, the term. “T_{1/2}” as used herein, refers to the time that it takes for progesterone blood concentration to decline to one-half of the maximum level.

Collectively AUC, Cₘₐₓ, and optionally Tₘₐₓ and T_{1/2}, are the principle pharmacokinetic parameters that can characterize the pharmacokinetic responses of a particular drug product such as progesterone in an animal or human subject.

### DESCRIPTION

Provided herein are oral pharmaceutical compositions comprising solubilized or partially solubilized progesterone. Further disclosed herein are data demonstrating the efficacy of these pharmaceutical compositions, as well as methods of using the described pharmaceutical compositions. Generally, the pharmaceutical compositions disclosed herein can be useful in mitigating the symptoms and effects of increased, decreased, or irregular estrogen levels.

Additional aspects and embodiments of this disclosure include: providing increased patient ease of use while potentially minimizing certain side effects from erroneous use, providing reduced metabolic and vascular side effects of commonly used synthetic progesterone, providing reduced food and allergy effects, providing improved bioavailability of progesterone as compared to the PROMETRIUM®, and in some embodiments providing for improved bioavailability of progesterone or a bioequivalent progesterone product at a reduced dose of API compared to the RLDs.

Various embodiments are improvements over exiting progesterone formulations, treatments, and methods of using these formulations and treatments. While not bound by theory, the elements of the pharmaceutical compositions of this disclosure provide improved bioavailability, improved pharmacokinetics, bioequivalent pharmaceutical compositions, and the potential to reduce the administered dosage strength. Bioavailability comparisons to commercially available forms, such as tablet and capsule forms, may be determined by standard pharmacokinetic techniques.

In embodiments, progesterone is solubilized or partially solubilized (partially suspended) when administered. The type of progesterone used, the form of that progesterone (i.e., solubilized or suspended), the different solubilizing agent used, the different excipients used, and the administration under proper conditions (i.e. fed, absence of concomitant medications, etc.) contribute, in part, to the improvements over existing progesterone compositions, methods, and treatments.

In embodiments, the pharmaceutical compositions do not include peanut oil.

In certain embodiments, the API is progesterone, which is solubilized or partially solubilized (partially suspended). In embodiments, progesterone is the sole API.

Generally, the pharmaceutical formulations described herein are prepared and administered as filled capsules, typically soft capsules or softgels of one or more materials well known in the art including, for example and without limitation, soft gelatin capsules. Ultra-micronized progesterone, as described herein, may also be prepared for administration in tablets or other well-known orally administered dosage forms using standard techniques.

In illustrative embodiments, total progesterone, i.e., dissolved and suspended progesterone, can be 20 to 50 wt %, e.g., 30 to 35 wt %, based on the weight of the entire fill, i.e., the liquid pharmaceutical formulation.

Other embodiments disclosed herein further provide more uniform dissolution of progesterone and reduced intra- and inter-patient PK parameters when compared to equal dosages of PROMETRIUM. Dissolution uniformity of progesterone in a formulation of this disclosure compared to PROMETRIUM at equal dosage strengths and using the same USP apparatus can be determined using standard techniques established for API dissolution testing, including that which is described in the examples below.

According to the PROMETRIUM prescribing information, progesterone absorption is highly variable from patient to patient and within the same patient. A clinical trial involving postmenopausal women who were administered PROMETRIUM once a day for five days resulted in the mean PK parameters listed in the following table:

| | PROMETRIUM Capsules Daily Dose | | |
|---|---|---|---|
| Parameter | 100 mg | 200 mg | 300 mg |
| Cₘₐₓ (ng/ml) | 17.3 +/− 21.9 | 38.1 +/− 37.8 | 60.6 +/− 72.5 |
| Tₘₐₓ (hr) | 1.5 +/− 0.8 | 2.3 +/− 1.4 | 1.7 +/− 0.6 |
| AUC₀₋₁₀ | 43.4 +/− 30.8 | 101.2 +/− 66.0 | 175.7 +/− 170.3 |
| (ng × hr/ml) | | | |

These values are highly variable as demonstrated by their standard deviations which, in some cases, exceed 100% of the noted mean value. In particular illustrative aspects and embodiments of this invention, it is possible, though not necessary, to reduce the standard deviations in one or more of these PK parameters.

Reduced intra- and inter-patient variability of progesterone according to this disclosure compared to PROMETRIUM can be assessed using techniques known to those of ordinary skill in the art and described elsewhere herein.

Other aspects of this disclosure include the use of formulations as described herein wherein progesterone is at least one API in said formulation for the treatment of an animal, especially a mammal, including humans: for endometrial hyperplasia; for secondary amenorrhea; as a method of treatment for preterm birth, when said animal has a shortened cervix, and other disease states or conditions treated with supplemental progesterone (collectively, “Progesterone-deficient States”) in a subject in need of treatment, and with a non-toxic effective amount of said formulations.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

For purposes of this disclosure, “prophylaxis” refers to administration of the progesterone, to an animal, especially a mammal, and in particular a human, to protect the animal from any of the disorders set forth herein, as well as others, before or after the disorder has occurred in the subject.

Exemplary dosage strengths for progesterone for use in the formulations described herein include, without limitation, 25 mg, 50 mg, 75 mg, 100 mg, 125 mg, 150 mg, 175 mg, 200 mg, 250 mg, 300 mg, 350 mg and 400 mg. In embodiments, progesterone dosage strength is from at least 25 mg to at least 200 mg. Specific dosage embodiments contain at least: 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, or 200 mg of progesterone per capsule.

In certain embodiments, the pharmaceutical compositions can contain at least about 50 mg, 75 mg, 100 mg, 150 mg, or 200 mg of progesterone. In certain embodiments, the pharmaceutical compositions contain from about 25 mg to about 50 mg, from about 75 mg to 100 mg, from about 50 mg to about 100 mg, about 75 mg, about 150 mg, about 200 mg, from about 100 mg to 150 mg, from about 150 mg to 200 mg, from 100 mg to 200 mg of progesterone. The lowest clinically effective dose of progesterone is used for treatment symptoms occurring due to irregular or inadequate hormone production, or for estrogen HRT patients. In one embodiment, the progesterone dosage is about 75 mg. In another embodiment, the progesterone dosage is about 150 mg. In another embodiment, the progesterone dosage is about 200 mg. In particular embodiments, the dosage is 75 mg, 150 mg, or 200 mg.

Solubilized compositions of this disclosure can be formulated for administration using techniques disclosed herein, and also using techniques well known in the art. Thus, an illustrative embodiment of a pharmaceutical composition of the invention comprises progesterone, at least 75% of the progesterone being solubilized (the balance being suspended/ultra-micronized as discussed elsewhere herein), and an oil, wherein the oil is medium chain fatty acid mono- and di-esters of one or more glycols, with or without surfactant.

In other embodiments, the progesterone in the pharmaceutical compositions is not more than about 20% solubilized, not more than about 19% solubilized, not more than about 18% solubilized, not more than about 17% solubilized, not more than about 16% solubilized, not more than about 15% solubilized, not more than about 14% solubilized, not more than about 13% solubilized, not more than about 12% solubilized, not more than about 11% solubilized, not more than about 10% solubilized, not more than about 9% solubilized, not more than about 8% solubilized, not more than about 7% solubilized, not more than about 6% solubilized, or not more than about 5% solubilized, with the balance being suspended in the formulation as discussed elsewhere herein. The suspended/ultra-micronized progesterone is absorbable by the body and retains biological functionality despite not being soluble in the formulation. In a particular embodiment, the progesterone is about 15% solubilized in the formulation, with balance (about 85%) being suspended/ultra-micronized. In another embodiment, the progesterone is about 5% solubilized in the formulation, with balance (about 95%) being suspended/ultra-micronized.

In certain embodiments, progesterone solubility in various solubilizing agents ranges from 27 mg/g to 95 mg/g. More specifically, in certain embodiments, progesterone's solubility in solubilizing agents is from 27.8 mg/g, 57.4 mg/g, 70.5 mg/g, 73.4 mg/g, 86.4 mg/g, to 95 mg/g.

Progesterone may be micronized/ultra-micronized via any one of the multiple methods typically utilized by the ordinarily skilled artisan.

Particle size may be determined in any suitable manner. For example, a Beckman Coulter LS 13 320 Laser Diffraction Particle Size Analyzer (the “Beckman Device”) may be used to determine particle size. Particle size may be represented by various metrics, for example, through an X50 particle size, or X90 particle size, or similar descriptions of particle size.

The Beckman Device may be used with various modules for introducing a sample for analysis. The Beckman Device may be used with the LS 13 320 Universal Liquid Module (“ULM”). The ULM is capable of suspending samples in the size range of 0.017 μm to 2000 μm. The ULM is a liquid based module that allows for delivery of the sample to the sensing zone. The ULM recirculates the sample through the Beckman Device. The ULM comprises two hoses, one for fluid delivery and another for waste. The total volume used may be 125 mL or less. A sample mass of from about 1 mg to about 10 g may be used. The ULM may interact with the Beckman Device via pins that fit into slots on the ULM. The ULM may use a variety of suspension fluids, for example, water, butonol, ethanol, chloroform, heptanes, toluene, propanol, COULTER Type 1B Dispersant (“Coulter IB”), and a variety of other suspension fluids. Surfactants may also be used, though pump speed should be adjusted to prevent excessive bubbling. Coulter 1B may comprise one or more of acetaldehyde, ethylene oxide, or 1,4-dioxane. The Beckman Device may be configured to use a variety of optical theories, including the Fraunhofer optical model and the Mie Theory.

The Beckman Device may comprise software to control the Beckman Device while the ULM is in use. The software may control, for example, pump speed, use of de-bubble routine, rinse routine, sonicate routine, and fill routine, among others. Parameters regarding the sample run may also be configured. For example, run length may be set. Though any suitable run length may be used, in various embodiments, a time period of 30 seconds to 120 seconds, and preferably between 30 seconds and 90 seconds may be used.

The Beckman Device may be used with the LS 13 320 Micro Liquid Module (“MLM”). The MLM is capable of suspending samples in the size range of 0.4 μm to 2000 μm. The MLM is a liquid based module that allows for delivery of the sample to the sensing zone. The MLM includes a stirrer. The total volume used may be 12 mL or less. The MLM may use a variety of suspension fluids, both aqueous and non-aqueous.

In various embodiments, ultra-micronized progesterone has an X50 value of less than about 15 microns, less than about 10 microns, less than about 5 microns or less than about 3 microns; and an X90 value of less than about 25 microns, less than about 20 microns, or less than about 15 microns.

In various embodiments, ultra-micronized progesterone is formulated with peanut and peanut-oil free excipients.

Solvent System

In various embodiments, a solvent system solubilizes one or more APIs, and in particular, progesterone. The solvent system is a mixture of solubilizing agents, together with co-solvents, surfactants, or other excipients. In certain embodiments, the solvent system comprises non-toxic, pharmaceutically acceptable solvents (alternatively referred to as “carriers”), co-solvents, surfactants, and excipients suitable for oral administration or absorption.

In embodiments, oils having medium chain fatty acids as a predominant or majority component are used as solubilizing agents/carriers to solubilize the one or more APIs. In certain embodiments, the solubilizing agents comprise medium chain fatty acid esters (e.g., esters of glycerol, ethylene glycol, or propylene glycol) or mixtures thereof. In certain embodiments, the medium chain fatty acids comprise chain lengths from C6 to C14. In certain embodiments the medium chain fatty acids comprise chain lengths from C6 to C12. In still other embodiments, the medium chain fatty acids are mono-, di-, or triglycerides predominately with chain lengths from C8 to C10. As noted elsewhere herein, the medium chain fatty acids can be saturated. In certain embodiments, the medium chain fatty acids are predominantly saturated, i.e., greater than about 60%, greater than about 70%, greater than about 75%, greater than about 80%, greater than about 85%, greater than about 90%, or greater than about 95% saturated. In particular embodiments, the solubilizing agent comprises a mixed triglyceride predominantly comprising C8 and C10 fatty acids. In other particular embodiments, the solubilizing agent comprises both simple and mixed triglycerides predominately comprising C8 and C10 fatty acids. In particular embodiments, the solubilizing agent comprises a mixed triglyceride predominantly comprising saturated C8 and C10 fatty acids. In other particular embodiments, the solubilizing agent comprises both simple and mixed triglycerides predominately comprising saturated C8 and C10 fatty acids.

In some embodiments, the solubilizing agent/carrier is selected to enhance dissolution or suspension of progesterone. In further various embodiments, the solubilizing agent/carrier is selected to enhance absorption of the API by cells of a mammal. For example, certain carriers may be selected to enhance absorption of the other formulation components, including the API. Absorption may comprise absorption into any cell and particularly absorption into digestive system cells, such as intestinal cells, and cells of the female reproductive system, such as the vagina and the cervix. Selected mono-, di-, or triglyercides are particularly suited to aid in cellular absorption.

In certain embodiments, a surfactant is used to aid in solubilizing, partially solubilizing, or suspending progesterone in the solubilizing agent. For example, a surfactant, such as GELUCIRE 44/14, can be used. In certain embodiments, GELUCIRE 44/14 may be heated to approximately 45-50° C. When the surfactant is completely melted, it is added to an appropriate container that contains the solubilizing agent. The solubilizing agent and surfactant are mixed. During this mixing process the progesterone is added, thus, solubilizing, partially solubilizing, or suspending progesterone. In certain embodiments, the solubilizing agent is liquid at between room temperature and about 50° C., at or below 50° C., at or below 40° C., or at or below 30° C.

In various embodiments, the solubilizing agent/carrier can be an oil having medium chain fatty acids as a majority or predominant component. Suitable medium chain fatty acids include caproic acid (C6), enanthic acid (C7), caprylic acid (C8), pelargonic acid (C9), capric acid (C10), undecylic acid (CI 11), lauric acid (C12), tridecylic acid (C13), and myristic acid (C14). In use, these fatty acids are predominantly saturated (e.g., greater than 50%, greater than about 60%, greater than about 70%, greater than about 80%, greater than about 90%, or greater than about 95%, or about 100%). In certain embodiments, predominantly C6 to C12 saturated fatty acids are contemplated. In certain embodiment, predominaely C8 to C10 saturated fatty acids are contemplated. In certain embodiments, these fatty acids may be bound to glycerin, propylene glycol, ethylene glycol, or polyethylene glycol. In certain embodiments, the solubilizing agent is selected from at least one of a solvent or co-solvent.

In particular embodiments, the solubilizing agent can comprise a mixture of caprylic/capric triglycerides; caproic/caprylic/capric/lauric triglycerides; caprylic/capric/linoleic triglycerides; caprylic/capric/succinic triglycerides; propylene glycol dicaprylate/dicaprate; and combinations and derivatives thereof. In further embodiments, in addition to the various mixtures of the specified triglycerides, the solubilizing agent can further include polyethylene glycol.

Suitable carriers/solubilizing agents further include esters of saturated coconut and palm kernel oil and derivatives thereof, including fractionated coconut oils and palm kernel oils; and triglycerides of fractionated vegetable fatty acids, and derivatives thereof and combinations thereof. In further various embodiments, the carrier/solubilizing agent may comprise one or more monoglycerides, diglycerides, triglycerides, and combinations thereof having predominately C6-C12 fatty acid esters. Specifically contemplated as the solvent are mono-, di-, and triglycerides of saturated C8-C10 (caprylic/capric) fatty acids. Exemplary glycerin based solubilizing agents include MIGLYOLs®, which are caprylic/capric triglycerides (SASOL Germany GMBH, Hamburg). MIGLYOLs includes MIGLYOL 810 (caprylic/capric triglyceride), MIGLYOL 812 (caprylic/capric triglyceride), MIGLYOL 816 (caprylic/capric triglyceride), and MIGLYOL 829 (caprylic/capric/succinic triglyceride). Other caprylic/capric triglyceride solubilizing agents are likewise contemplated, including, for example: caproic/caprylic/capric/lauric triglycerides; caprylic/capric/linoleic triglycerides; caprylic/capric/succinic triglycerides. In certain embodiments, CAPMUL MCM, medium chain mono- and di-glycerides of caprylic/capric fatty acids, is the solubilizing agent. In other embodiments, CAPMUL PG-8 (Propylene Glycol Monocaprylate), CAPMUL PG-10 (Propylene Glycol Monocaprate), or other caprylic/capric CAPMULs is the solubilizing agent. Triglycerides of fractionated vegetable fatty acids, and combinations or derivatives thereof can be the solubilizing agent, in certain embodiments.

Additional examples of solubilizing agents include a polyethylene glycol glyceride (Gelucire®; GATTEFOSSE SAS, Saint-Priest, France); a propylene glycol; a caproic/caprylic/capric/lauric triglyceride; a caprylic/capric/linoleic triglyceride; a caprylic/capric/succinic triglyceride; propylene glycol monocaprylate; propylene glycol monocaprate; (Capmul® PG-8 and 10; the CAPMUL brands are owned by ABITEC, Columbus Ohio); propylene glycol dicaprylate; propylene glycol dicaprylate; a diethylene glycol mono ester (including 2-(2-Ethoxyethoxy)ethanol (also referred to as TRANSCUTOL®); diethylene glycol monoethyl ether; esters of saturated coconut and palm kernel oil and derivatives thereof; triglycerides of fractionated vegetable fatty acids, and combinations and derivatives thereof.

In other aspects and embodiments, progesterone is fully solubilized using, for example and without limitation, sufficient amounts of: TRANSCUTOL and MIGLYOL; TRANSCUTOL, MIGLYOL and CAPMUL PG-8 or CAPMUL PG-10; CAPMUL MCM (Medium Chain Mono- and Diglycerides); CAPMUL MCM and a non-ionic surfactant; and CAPMUL MCM and GELUCIRE.

In particular embodiments, the solubilizing agent comprises combinations of mono- and di-esters of propylene glycol or ethylene glycol or mono-, di-, and triglyceride combinations.

In certain embodiments, polyethylene glycol glyceride (GELUCIRE®, GATTEFOSSÉ SAS, Saint-Priest, France) can be used as the solubilizing agent or as a surfactant. For example, GELUCIRE 44/14 can be used. GELUCIRE 44/14 is a non-ionic water dispersible surfactant, also known as lauroyl macrogol-32 glycerides EP and lauroyl polyoxyl-32 glycerides NF. For example, in certain embodiments, a non-ionic surfactant is selected from one or more of glycerol and polyethylene glycol esters of long chain fatty acids, such GELUCIRE 44/14 (discussed previously herein), GELUCIRE 44/11, GELUCIRE 39/01 (glycerol esters of saturated C12-C18 fatty acids), GELUCIRE 43/01 (hard fat NF/JPE), GELUCIRE 50/13 (stearoyl macrogol-32 glycerides EP, stearoyl polyoxyl-32 glycerides NF, and stearoyl polyoxylglycerides (USA FDA IIG)). These surfactants may be used at concentrations greater than about 0.01 wt. %, and typically in various amounts of about 0.01 wt. %; about 10.0 wt. %; about 10.1 wt. %; about 20 wt. %; about 20.1 wt. %; and about 30 wt. %. More specifically, these surfactants may be used at concentrations between 0.01 wt. % to 5.00 wt. %.

Other non-ionic surfactants include, for example and without limitation one or more of oleic acid, linoleic acid, palmitic acid, and stearic acid. In other embodiments, non-ionic surfactants can comprise polyethylene sorbitol esters, such as polysorbate 80, which is commercially available under the trademark TWEEN 80 (polysorbate 80) (Sigma Aldrich, St. Louis, Mo.). Polysorbate 80 comprises approximately 60%-70% oleic acid with the remainder comprising primarily linoleic acids, palmitic acids, and stearic acids. Polysorbate 80 may be used in amounts ranging from about 5 to 50%/of the pharmaceutical composition by mass, and in particular embodiments, about 30% of the pharmaceutical composition total mass.

Yet another non-ionic surfactants is PEG-6 palmitostearate and ethylene glycol palmitostearate, which is available commercially as TEFOSE® 63 (GATTEFOSSÉ SAS, Saint-Priest, France), which can be used with, for example, CAPMUL MCM having ratios of MCM to TEFOSE 63 of, for example, 8:2 or 9:1. In other embodiments, other solubilizing agents/non-ionic surfactants combinations include, for example, MIGLYOL 812:GELUCIRE 50/13 or MIGLYOL 812:TEFOSE 63.

In still further embodiments, the surfactant can be an anionic surfactant, for example: ammonium lauryl sulfate, dioctyl sodium sulfosuccinate, perfluoro-octane sulfonic acid, potassium lauryl sulfate, or sodium stearate.

In certain embodiments, the non-ionic or anionic surfactant(s) can be used alone with at least one solubilizing agent or can be used in combination with other surfactants. Accordingly, such surfactants, or any other excipient as set forth herein, may be used to solubilize one or more APIs. In this disclosure, the API is progesterone. The combination of solubilizing agent, surfactant, and other excipients should be designed whereby the one or more APIs are delivered to the target tissue and result the intended effect of the API.

Various ratios of the noted solubilizing agents can be used for suspension or solubilization of progesterone. CAPMUL MCM and a non-ionic surfactant, e.g., GELUCIRE 44/14 (Lauroyl macrogol-32 glycerides EP Lauroyl polyoxyl-32 glycerides NF Lauroyl polyoxylglycerides (USA FDA IIG)), can be used at ratios of about 9:1, 7:3, 6:4, and 6:3 when progesterone is the sole API and at ratios of 65:35, 70:30, 75:25, 80:20, 85:15 and 90:10 with estradiol as the sole API. Other non-limiting examples include CAPMUL MCM and GELUCIRE 44/14 used in ratios including, for example, and without limitation, 99:1 to 2:1, including, for example and without limitation: 60:40, 65:35, 70:30, 75:25, 80:10, 80:15, 85:20, 90:10, and 98:1; CAPMUL MCM and GELUCIRE 39/01 can be used in ratios including, for example and without limitation, 6:4, 7:3, and 8:2 (one or more API composition); CAPMUL MCM and GELUCIRE 43/01 can be used in ratios including, for example and without limitation, 7:3, and 8:2 (one or more API composition); and CAPMUL MCM and GELUCIRE 50/13 can be used in ratios including, for example and without limitation, 7:3, and 8:2, and 9:1. In other embodiments, CAPMUL MCM and GELUCIRE were used in ratios of up to about 65:1, e.g., 8:1, 22:1, 49:1, 65:1 and 66:1. Thus, useful ratios can be, e.g., 8:1 or greater, e.g., 60 to 70:1.

Combinations of these solubilizing agents can produce solubilized or partially solubilized progesterone, depending upon the desired unit dosage amount of progesterone. The greater the amount of progesterone per unit dosage form, the less progesterone may be solubilized. The upward limit of dosage strength per unit dose it generally limited only by the practical size of the final dosage form.

In illustrative embodiments, solubilizing agents used to suspend, partially solubilize, or fully solubilize progesterone include medium chain fatty acid esters, (e.g., esters of glycerol, ethylene glycol, polyethylene glycol, or propylene glycol) and mixtures thereof. In illustrative embodiments, the medium chain fatty acids are C6 to C14 or C6 to C12 fatty acids. In illustrative embodiments, the medium chain fatty acids are saturated, or predominantly saturated, e.g., greater than about 60% or greater than about 75% saturated. In illustrative embodiments, progesterone is soluble in the oils at room temperature, although it may be desirable to warm certain oils initially during manufacture to improve viscosity. In illustrative embodiments, the oil or oil/surfactant is liquid at between room temperature and about 50° C., e.g., at or below 50° C., at or below 40° C., or at or below 30° C. In illustrative embodiments, GELUCIRE 44/14 is heated to about 65° C. and CAPMUL MCM is heated to about 40° C. to facilitate mixing of the oil and non-ionic surfactant, although such heating is not necessary to dissolve the estradiol or progesterone.

In illustrative embodiments, the solubility of estradiol in the solubilizing agent or combination of solubilizing agents is at least about 0.5 wt %, e.g., 0.8 wt % or higher, or 1.0 wt % or higher. Illustrative examples of mono- and diglycerides of medium chain fatty acids include, among others, CAPMUL MCM, CAPMUL MCM C10 (Glyceryl Monocaprate), CAPMUL MCM C8 (Glyceryl Monocaprylate), and CAPMUL MCM C8 EP (Glyceryl Monocaprylate). These oils are C8 and C10 fatty acid mono- and diglycerides. Illustrative examples of oils that are triglycerides of medium chain fatty acids include, among others, MIGLYOL 810 and MIGLYOL 812.

Illustrative examples of solubilizing agents that are medium chain fatty acid esters of propylene glycol include, among others, CAPMUL PG-8, CAPMUL PG-2L EP/NF (Propylene Glycol Dilaurate), CAPMUL PG-8 NF (Propylene Glycol Monocaprylate), CAPMUL PG-12 EP/NF (Propylene Glycol Monolaurate) and CAPRYOL (Propylene glycol monocaprylate (type II) NF). Other illustrative examples include MIGLYOL 840 (Propylene Glycol Dicaprylate/Dicaprate).

Illustrative examples of solubilizing agents that are medium chain fatty acid esters of polyethylene glycol include, among others, GELUCIRE 44/14 (PEG-32 glyceryl laurate EP), which is polyethylene glycol glycerides composed of mono-, di- and triglycerides and mono- and diesters of polyethylene glycol. Without intending to be bound to any particular mechanism, it appears that at least in formulations comprising small amounts of GELUCIRE, e.g., 10 wt % or less, the primary function of this oil is as a non-ionic surfactant.

These illustrative examples comprise predominantly medium chain length, saturated, fatty acids, specifically predominantly C8 to C12 saturated fatty acids. In particular embodiments, the predominantly C8 to C12 saturated fatty acids comprise not less than 50 wt %, not less than 75 wt %, not less than 85 wt %, not less than 90 wt %, or not less than 95 wt % of the solubilizing agent.

It will be understood that commercially available fatty acid esters of glycerol and other glycols are often prepared from natural oils and therefore may comprise components additional to the fatty acid esters that comprise the predominant (by weight) component(s) and that therefore are used to characterize the product. Such other components may be, e.g., other fatty acid triglycerides, mono- and diesters, free glycerol, or free fatty acids. So, for example, when an oil/solubilizing agent is described herein as a saturated C8 fatty acid mono- or diester of glycerol, it will be understood that the predominant component of the oil, i.e., >50 wt % (e.g., >75 wt %, >85 wt % or >90 wt %) are caprylic monoglycerides and caprylic diglycerides. For example, the Technical Data Sheet by ABITEC for CAPMUL MCM C8 describes CAPMUL MCM C8 as being composed of mono and diglycerides of medium chain fatty acids (mainly caprylic) and describes the alkyl content as <=1% C6, >=95% C8, <=5% C10, and <=1.5% C12 and higher.

By way of further example, MIGLYOL 812 is generally described as a C8-C10 triglyceride because the fatty acid composition is at least about 80% caprylic (C8) acid and capric (C10) acid. However, it can also comprise small amounts of other fatty acids, e.g., less than about 5% of caproic (C6) acid, lauric (C12) acid, and myristic (C14) acid.

Specifically, a product information sheet for MIGLYOL by SASOL provides the composition of fatty acids as follows:

| Tests | 810 | 812 | 818 | 829 | 840 |
|---|---|---|---|---|---|
| Caproic acid | max. 2.0 | max. 2.0 | max. 2 | max. 2 | max. 2 |
| (C6:0) | | | | | |
| Caprylic acid | 65.0-80.0 | 50.0-65.0 | 45-65 | 45-55 | 65-80 |
| (C8:0) | | | | | |
| Capric acid | 20.0-35.0 | 30.0-45.0 | 30-45 | 30-40 | 20-35 |
| (C10:0) | | | | | |
| Lauric acid | max. 2 | max. 2 | max. 3 | max. 3 | max. 2 |
| (C12:0) | | | | | |
| Myristic acid | max. 1.0 | max. 1.0 | max. 1 | max. 1 | max. 1 |
| (C14:0) | | | | | |
| Linoleic acid | — | — | 2-5 | — | — |
| (C18:2) | | | | | |
| Succinic acid | — | — | — | 15-20 | — |

Where certain embodiment of this invention are described as comprising (or consisting essentially of) a capsule shell, estradiol solubilized in C8-C10 triglycerides, and a thickening agent, it will be understood that the fatty acid esters component of the formulation may be, e.g., MIGLYOL 812 or a similar product.

By way of further illustration, GELUCIRE 44/14 is generally described as lauroyl polyoxyl-32 glycerides, i.e., polyoxyethylene 32 lauric glycerides (which is a mixture of mono-, di-, and triesters of glycerol and mono- and diesters of PEGs) because the fatty acid composition is 30 to 50% lauric acid and smaller amounts of other fatty acids, e.g., up to 15% caprylic acid, up to 12% capric acid, up to 25% myristic acid, up to 25% palmitic acid, and up to 35% stearic acid. The product may also contain small amounts of non-esterified glycols.

Similarly, where certain embodiment of this invention are described as comprising (or consisting essentially of) a capsule shell, estradiol solubilized in triglycerides, and a thickening agent that is a non-ionic surfactant comprising PEG-6 stearate, ethylene glycol palmitostearate, and PEG-32 stearate, it will be understood that the thickening agent component of the formulation may be, e.g., TEFOSE 63 (PEG-6 palmitostearate and ethylene glycol palmitostearate) or a similar product.

In illustrative embodiments of the invention, the selected solubilizing agent does not require excessive heating in order to solubilize progesterone. For example, when the formulation comprises medium chain fatty acid mono- and diglycerides (e.g., CAPMUL MCM) and polyethylene glycol glycerides (e.g., GELUCIRE) as a surfactant, the oil or the surfactant can be warmed up, e.g., to about 65 C in the case of the surfactant and less in the case of the oil, to facilitate mixing of the oil and surfactant. The progesterone can be added as the mixture cools, e.g., to below about 40 C or to below about 30 C, even down to room temperature.

In various embodiments, a lubricant is used. Any suitable lubricant may be used, such as, for example and without limitation, lecithin, and in various embodiments, a mixture of polyethylene glycol (“PEG”) esters, glycerides, and PEG, such as is commercially available under the trade name GELUCIRE (Gattefosse, FR) may also be used as a lubricant. Suitable lubricants may also comprise calcium stearate, ethyl oleate, ethyl laureate, glycerin, glyceryl palmitostearate, hydrogenated vegetable oil, magnesium, oxide, magnesium stearate, poloxamer, glycols, and phospholipid mixtures. In particular, a mixture of polyethylene glycol esters, glycerides, and PEG such as GELUCIRE 44/14, may be used as a lubricant. GELUCIRE 44/14 is a non-ionic water dispersible surfactant, also known as lauroyl macrogol-32 glycerides EP and lauroyl polyoxyl-32 glycerides NF. In various embodiments, GELUCIRE 44/14 acts as a suspension agent.

In various embodiments, an antioxidant is used. Any suitable antioxidant may be used, such as, for example and without limitation, butylated hydroxytoluene, also commercially referred to as BHT. Butylated hydroxytoluene, a derivative of phenol, is lipophilic and is thus suited to being intermixed with ultra-micronized progesterone and carriers disclosed or contemplated herein.

For example, in various embodiments, a pharmaceutical formulation comprises about 20% to about 80% solubilizing agent by weight, about 0.1% to about 5% lubricant by weight, and about 0.01% to about 0.1% antioxidant by weight.

In certain embodiments, the pharmaceutical composition further comprises at least one thickening agent. Generally, a thickening agent is added when the viscosity of the pharmaceutical composition provides less than desirable absorption following administration. Examples of thickening agents include: hard fats; propylene glycol; a mixture of hard fat EP/NF/JPE, glyceryl ricinoleate, ethoxylated fatty alcohols (ceteth-20, steareth-20) EP/NF (available as OVUCIRE® 3460, GATTEFOSSÉ, Saint-Priest, France); a mixture of hard fat EP/NF/JPE, glycerol monooleate (type 40) EP/NF (OVUCIRE WL 3264; a mixture of hard fat EP/NF/JPE, glyceryle monooleate (type 40) EP/NF (OVUCIRE WL 2944); and a mixture of various hard fats (WITEPSOL®, Sasol Germany GmbH, Hamburg, Germany). In certain embodiments, the viscosity of pharmaceutical compositions in accordance with various embodiments may comprise from about 50 cps to about 1000 cps at 25° C. A person of ordinary skill in the art will readily understand and select from suitable thickening agents.

In other embodiments, the thickening agent is a non-ionic surfactant. For example, polyethylene glycol saturated or unsaturated fatty acid ester or diester is the non-ionic surfactant thickening agent. In some embodiments, the non-ionic surfactant comprises a polyethylene glycol long chain (C16-C20) fatty acid ester and further comprises an ethylene glycol long chain fatty acid ester, such as PEG-fatty acid esters or diesters of saturated or unsaturated C16-C18 fatty acids, e.g., oleic, lauric, palmitic, and stearic acids. In embodiments, the non-ionic surfactant comprises a polyethylene glycol long chain saturated fatty acid ester and further comprises an ethylene glycol long chain saturated fatty acid ester, such as PEG- and ethylene glycol-fatty acid esters of saturated C16-C18 fatty acids, e.g., palmitic and stearic acids. Such non-ionic surfactant can comprise PEG-6 stearate, ethylene glycol palmitostearate, and PEG-32 stearate, such as but not limited to TEFOSE 63.

In certain embodiments, the non-ionic surfactant used as a thickening agent is not hydrophilic and has good emulsion properties. An illustrative example of such surfactant is TEFOSE 63, which has a hydrophilic-lipophilic balance (HLB) value of about 9-10.

The selection and amount of hydrophilic polymer may be based on the selection and amount of solubilizing agent. The pharmaceutical composition can include a hydrophilic polymer but optionally excludes a gelling agent. In embodiments having a hydrogel, from about 5% to about 10% of the total mass may comprise the hydrophilic polymer. In further embodiments, hydrogels may be employed. A hydrogel may comprise chitosan, which swell in response to contact with water. In various embodiments, a cream pharmaceutical composition may comprise PEG-90M.

In addition to the above, the pharmaceutical compositions described herein can include one or more thermoreversible gels, typically of the hydrophilic nature including for example and without limitation, hydrophilic sucrose and other saccharide-based monomers (U.S. Pat. No. 6,018,033, which is incorporated herein by reference).

The choice of excipient will depend on factors such as, for example, the effect of the excipient on solubility and stability. Additional excipients used in various embodiments may include colorants, flavoring agents, taste-masking agents and preservatives. In certain embodiments, colorants, comprise about 0.1% to about 2% of the pharmaceutical composition by weight. In certain embodiments, preservatives in the pharmaceutical composition comprise methyl and propyl paraben, in a ratio of about 10:1, and at a proportion of about 0.005% and 0.05% by weight.

Generally, the solubilizing agents, excipients, other additives used in the pharmaceutical compositions described herein, are non-toxic, pharmaceutically acceptable, compatible with each other, and maintain stability of the pharmaceutical composition and the various components with respect to each other. Additionally, the combination of various components that comprise the pharmaceutical compositions will maintain will result in the desired therapeutic effect when administered to a subject.

The choice of excipient will, to a large extent, depend on factors such as the particular mode of administration, the effect of the excipient on solubility and stability, and the nature of the dosage form. Excipients used in various embodiments may include colorants, flavoring agents, preservatives and taste-masking agents. Colorants, for example, may comprise about 0.1% to about 2% by weight. Preservatives may comprise methyl and propyl paraben, for example, in a ratio of about 10:1, and at a proportion of about 0.005% and 0.05% by weight.

As is with all oils, solubilizers, excipients and any other additives used in the formulations described herein, each is to be non-toxic and pharmaceutically acceptable.

As referenced above, the formulations of this disclosure are generally orally administered, typically via, for example, capsules such as soft capsules.

In certain embodiments, a pharmaceutical composition of this disclosure comprises progesterone, (with about 15% or less, and in particular embodiments, about 5% or less of the progesterone being solubilized—the balance being ultra-micronized/suspended as discussed elsewhere herein), and an oil, wherein the oil is medium chain fatty acid mono- and diesters of one or more glycols, with or without surfactant.

Pharmaceutical formulations in accordance with various embodiments comprise ultra-micronized progesterone. In further embodiments, a pharmaceutical formulation comprises ultra-micronized progesterone, a carrier, and a lubricant. In still further embodiments a pharmaceutical formulation comprises ultra-micronized progesterone, a carrier, a lubricant, and optionally an antioxidant. In still further embodiments, a pharmaceutical formulation comprises ultra-micronized progesterone, and a medium chain triglyceride as a carrier. In still further embodiments, a pharmaceutical formulation comprises ultra-micronized progesterone, and mono-, di-, or triglycerides of caprylicicapric acid as a carrier. Various further embodiments also comprise lecithin and optionally butylated hydroxytoluene.

In additional embodiments, a pharmaceutical formulation comprises ultra-micronized progesterone and at least one carrier, a lubricant, optionally an antioxidant, and other pharmaceutically acceptable excipients. For example, in various embodiments, a pharmaceutical formulation comprises about 20% to about 80% carrier by weight, about 0.1% to about 5% lubricant by weight, and about 0.01% to about 0.1% antioxidant by weight.

According to embodiments, a pharmaceutical formulation comprises ultra-micronized progesterone, at least one carrier, and a non-ionic surfactant.

The choice of excipient will, to a large extent, depend on factors such as the particular mode of administration, the effect of the excipient on solubility and stability, and the nature of the dosage form. Excipients used in various embodiments may include colorants, flavoring agents, preservatives, and taste-masking agents. Colorants, for example, may comprise about 0.1% to about 2% by weight. Preservatives may comprise methyl and propyl paraben, for example, in a ratio of about 10:1, and at a proportion of about 0.005% and 0.05% by weight.

In various embodiments, ultra-micronized progesterone is administered in a capsule. Capsules may be prepared using one or more film forming polymers. Suitable film forming polymers include natural polymers, such as gelatin, and synthetic film forming polymers, such as modified celluloses. Suitable modified celluloses include, but are not limited to, hydroxypropyl methyl cellulose, methyl cellulose.

Manufacturing

In certain embodiments, the pharmaceutical composition is prepared by blending progesterone with a pharmaceutically acceptable solubilizing agent, including for example and without limitation, at least one medium chain fatty acid such as medium chain fatty acids consisting of at least one mono-, di-, or triglyceride, or derivatives thereof, or combinations thereof. In particular embodiments, the pharmaceutical composition also comprises at least one glycol or derivatives thereof or combinations thereof or combinations of at least one glyceride and glycol. The glycol(s) may be used as solubilizing agents or to adjust viscosity and, thus, may be considered thickening agents. Other excipients can optionally be included, including, for example and without limitation, anti-oxidants, lubricants, and the like. In some embodiments, the pharmaceutical composition includes sufficient solubilizing agent(s) to fully solubilize the progesterone. It is expressly understood, however, that other volumes of solubilizing agent can be used depending on the level of progesterone solubilization desired. Persons of ordinary skill in the art will know and understand how to determine the volume of solubilizing agent and other excipients depending on the desired percent of progesterone to be solubilized in the pharmaceutical composition.

In illustrative embodiments, GELUCIRE 44/14 (lauroyl macrogol-32 glycerides EP, lauroyl polyoxyl-32 glycerides NF, lauroyl polyoxylglycerides (USA FDA IIG)) is heated to about 45-65° C. and CAPMUL MCM or MIGLYOL 812 is heated to about 40° C. to facilitate mixing of the oil and non-ionic surfactant, although such heating is not necessary to dissolve the progesterone.

Specific Examples disclosed herein provide additional principles and embodiments illustrating processes for manufacturing the pharmaceutical compositions disclosed herein.

Delivery Vehicle

The pharmaceutical compositions described herein can be delivered orally inside of a delivery vehicle, for example a capsule. In certain embodiments, the capsules are soft capsules made of materials well known in the pharmaceutical arts such as gelatin. In other embodiments, the delivery vehicle is integral with the pharmaceutical composition (i.e., the pharmaceutical composition is the delivery vehicle). Hard or soft shell capsules can be used to administer the API. In certain embodiments, capsules may be prepared by forming the two capsule halves, filling one of the halves with a fill solution, and then sealing the capsule halves together to form the finished capsule.

Hard shell capsules may be prepared by combining the “Body” and the “Cap”. The “Body” of the capsule is filled with the “fill mass” and then closed with the “Cap”. The “Body”/“Cap” interface is then sealed/banded.

Soft gelatin (“softgel”) capsules may be prepared using a rotary die encapsulation process, as further described below. Softgel capsules may contain the formulation disclosed herein as a “fill material.” The soft gelatin capsule do not contain one or more of the following as the fill material: hydrophilic gel-forming bioadhesive (e.g., mucoadhesive) agents; a lipophilic agent and a gelling agent for the lipophilic agent, or a hydrodispersible agent. In some embodiments, the hydrophilic gel-forming bioadhesive agent is carboxyvinylic acid; hydroxypropylcellulose; carboxymethylcellulose; gelatin; xanthane gum; guar gum; aluminum silicate; or mixtures thereof. In still other embodiments, the lipophilic agent is a liquid triglyceride; solid triglyceride (e.g., with a melting point of about 35° C.); carnauba wax; cocoa butter; or a mixture thereof. In certain embodiments, the gelling agent is a hydrophobic colloidal silica. And in still other embodiments, the hydrodispersible agent can be polyoxyethylene glycol; polyoxyethylene glycol 7-glyceryl-cocoate; or a mixture thereof.

The softgel capsule itself may comprise a gelatin material in a relatively solid or stiff form. The gel capsule defines an inner volume that contains the fill material. Dissolution of the gelatin material may commence at various points after administration, such as in the digestive tract (mouth, esophagus, stomach and intestines), or in another body cavity, such as the vaginal tract.

Gel capsules may be prepared using one or more film forming polymers. Suitable film forming polymers include, but are not limited to, natural polymers, such as gelatin, and synthetic film forming polymers, such as modified celluloses. Suitable modified celluloses include, but are not limited to, hydroxypropyl methyl cellulose, methyl cellulose.

Suitable shell additives, for either a hard or soft shell capsules, may include plasticizers, opacifiers, colorants, humectants, preservatives, flavorings, and buffering salts and acids, and combinations thereof. The main ingredients of the capsule shell is primarily gelatin (or a gelatin substitute for non-gelatin capsules), plasticizer, and purified water. Hard shell and soft shell capsules differ primarily in the amount of plasticizer present that is used in the capsule shell.

Plasticizers are chemical agents added to gelatin to make the material softer and more flexible. Suitable plasticizers include, but are not limited to, glycerin, sorbitol solutions which are mixtures of sorbitol and sorbitan, and other polyhydric alcohols such as propylene glycol and maltitol or combinations thereof.

Opacifiers are used to opacify the capsule shell when the encapsulated active agents are light-sensitive. Suitable opacifiers include titanium dioxide, zinc oxide, calcium carbonate and combinations thereof.

Colorants can be used for marketing and product identification/differentiation purposes. Suitable colorants include synthetic and natural dyes and combinations thereof.

Flavorings can be used to mask unpleasant odors and tastes of fill formulations. Suitable flavorings include synthetic and natural flavorings. The use of flavorings can be problematic due to the presence of aldehydes which can cross-link gelatin. As a result, buffering salts and acids can be used in conjunction with flavorings that contain aldehydes in order to minimize cross-linking of the gelatin.

In accordance with various embodiments, a softgel dosage form is used.

As the softgel dissolves, the inner volume may come into fluid communication with the digestive system, allowing the fill material to leach outside the softgel. A softgel may also be punctured, cut, or otherwise opened outside a body. The fill material may then be poured or squeezed outside the gel capsule and applied on or in the body, such as within the vaginal cavity.

Humectants can be used to suppress the water activity of the softgel. Suitable humectants include glycerin, sorbitol, propylene glycol, microcrystalline cellulose, silica, mineral oil, and combinations thereof which are often components of the plasticizer composition. Regulated water activity in pharmaceutical compositions and dosage forms, such as capsules, can improve the compatibility and stability of the compositions and forms. This is because when hydrolosis is regulated chemical degradation caused by water is also regulated (or slowed, as is desirable in the present case). Thus, by regulating water in the present compositions, the capsule shells are less likely to soften, dissolve, break, or leak during storage. Moreover, due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl esters (collectively known as “parabens”) or combinations thereof.

The fill material may comprise a liquid, such as an oil, a solution, a suspension, or other acceptable forms. The active ingredient or active ingredient may be contained within the liquid.

Hard and softgel capsules can be manufactured according to various techniques known in the art. In particular embodiments, softgel capsules can be prepare using a rotary die encapsulation process. An exemplary process is disclosed in Wilkinson, P. K. et al., 1990, “Softgels: manufacturing considerations.” In: Specialized Drug Delivery Systems, P. Tyle (Ed.), pp. 409-449, Marcel Dekker, Inc., New York, the entirety of which is hereby incorporated by reference.

In other embodiments, softgels can be prepared according to the process disclosed in PCT/US2000/005178, the entirety of which is incorporated herein by reference.

Hard shell capsules can also be used as the delivery vehicle. These capsules may be prepared by forming the two capsule halves, filling one half with the fill material, and then sealing the halves together to form the finished capsule. In other embodiments, hard shell capsules may be prepared by combining a “body” and a “cap.” The “body” of the capsule is filled with the fill material and then closed with the cap. The body/cap interface is then sealed or banded.

Drawings

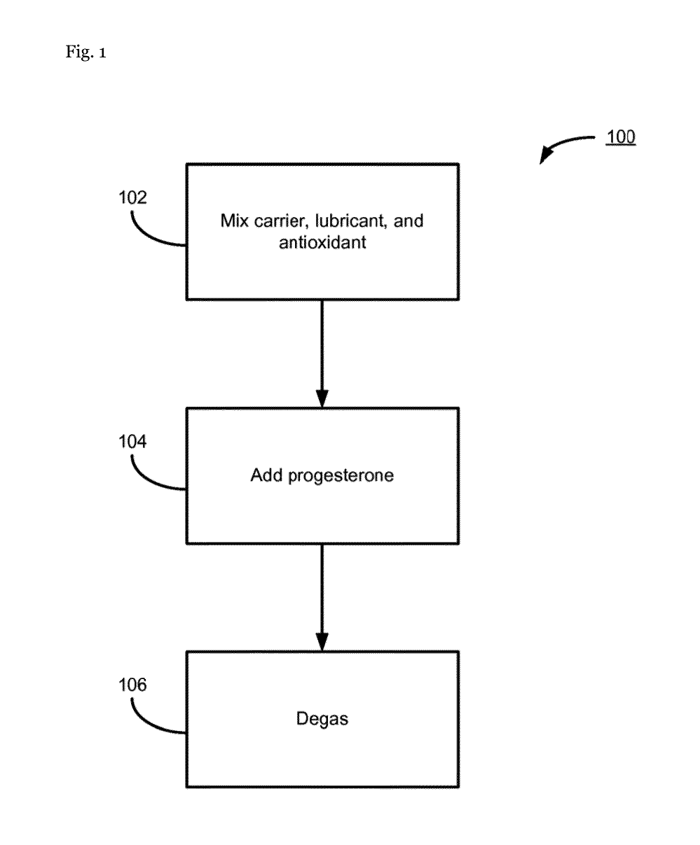

Methods of manufacture in accordance with various embodiments are shown in FIGS. 1-3. With reference to FIG. 1, method of fill material, i.e. fill mass, preparation 100 is shown. Operation 102 comprises mixing a solubilizing agent, a surfactant (i.e. lubricant), and an antioxidant as described herein. For example, lecithin and butylated hydroxytoluene may be mixed with one or more medium chain mono-, di- or triglycerides, or combinations thereof. Mixing may be facilitated by an impellor, agitator, or other suitable means. Operation 102 may be performed under an inert or relatively inert gas atmosphere, such as nitrogen gas N₂. Mixing may be performed in any suitable vessel, such as a stainless steel vessel.

Operation 104 may comprise mixing progesterone (progesterone) into the mixture of the solubilizing agent, the surfactant (i.e. lubricant), and the antioxidant. A pasty substance is thus formed. Mixing may occur in a steel tank or vat. Mixing may be facilitated by an impellor, agitator, or other suitable means. Operation 104 may be performed under an inert or relatively inert gas atmosphere, such as nitrogen gas N₂. Operation 106 comprises degasing. The resulting mixture from operation 106 may comprise a pharmaceutical composition suitable for production into a softgel capsule.

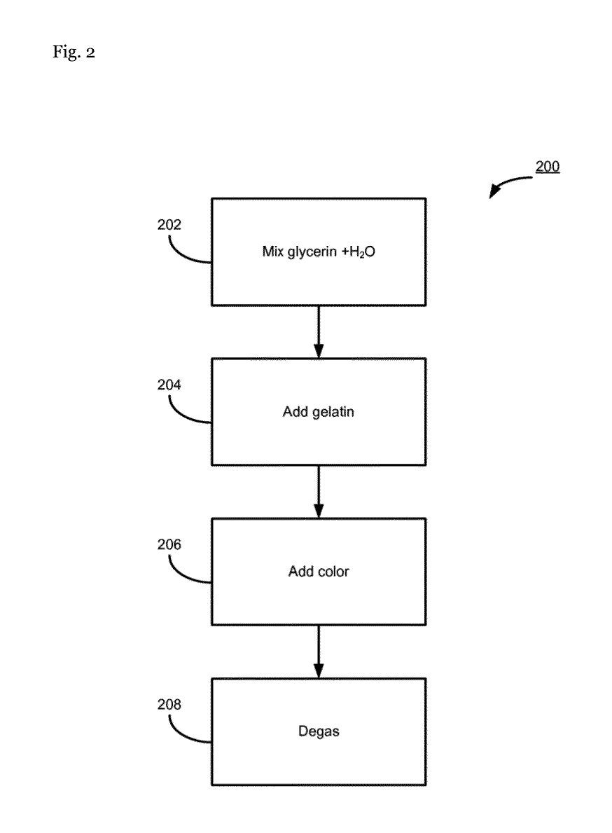

With reference to FIG. 2, softgel capsule, i.e., gel mass, production 200 is shown. Operation 202 comprises mixing glyercin with water. The water used in operation 202 may be purified by any suitable means, such as reverse osmosis, ozonation, filtration (e.g., through a carbon column) or the like. Mixing may be facilitated by an impellor, agitator, or other suitable means. Operation 202 may be performed under an inert or relatively inert gas atmosphere, such as nitrogen gas N₂. Heating may be performed until the temperature reaches 80°±5° C.

Operation 204 comprises the addition of gelatin to the glycerin water mixture. Mixing may be facilitated by an impellor, agitator, or other suitable means. Operation 204 may be performed under an inert or relatively inert gas atmosphere, such as nitrogen gas N₂. A vacuum may be drawn in operation 204 to de-aerate.

Operation 206 comprises addition of an excipient (i.e. coloring agent) such as a dye. A coloring agent may comprise products sold under the trademark OPATINT or the suitable agent. Operation 206 may be performed under an inert or relatively inert gas atmosphere, such as nitrogen gas N₂. Operation 208 comprises degasing. The resulting mixture from operation 208 may comprise a gel capsule material suitable for use as a gel capsule in production of a softgel capsule.

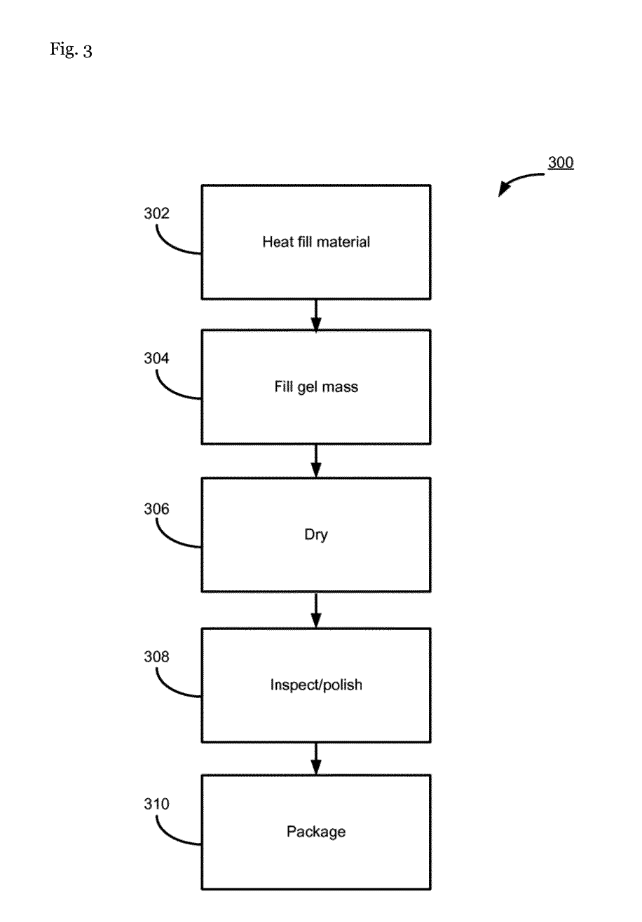

With reference to FIG. 3, softgel capsule assembly process 300 is shown. Operation 302 comprises heating the fill material. The pharmaceutical composition may be heated to any suitable temperature. In various embodiments, the pharmaceutical composition is heated to 30° C.+/−3° C. pharmaceutical composition maybe heated in a fill hopper. A fill hopper may comprise a device configured to hold a volume of the pharmaceutical composition or to dispense the pharmaceutical composition in controlled volumes.

Operation 304 comprises filling a gel mass. A gel mass may be taken from the gel capsule material produced in operation 208 of FIG. 2. Filling may be performed by injecting, placing, or otherwise disposing the pharmaceutical composition within a volume defined by the gel capsule material. The filling may occur in an encapsulator. The spreader boxes may be a temperature of 55° C.+/−10° C. The wedge temperature may be 38° C.+/−3° C. The drum cooling temperature may be 4° C.+/−2° C. The encapsulator may be lubricated using MIGLYOL 812. Operation 304 thus produces one or more softgel capsules. Filling may comprise producing a ribbon of thickness 0.85±0.05 mm using spreader box knobs. The pharmaceutical composition may be injected into the gel to produce a fill weight having target weight ±5% (i.e., 650±33 mg and 325±16.3 mg).

Operation 306 comprises drying the softgel capsules. Drying may be performed in a tumble dryer, tray dryer, or combinations thereof. For example, drying may be performed in a tumble drying basket for between about 10 minutes and about 120 minutes. Drying may continue in a drying room for about 24 hours to about 72 hours. Polishing may be performed with isopropyl alcohol.

Design Factors for Encapsulated Pharmaceutical Compositions

In certain embodiments, the pharmaceutical composition is designed to maximize API solubility, and other favorable characteristics without sacrificing efficacy, while simultaneously improving bioavailability in subjects. Other favorable characteristics, besides improving bioavailability as compared to the RLD, include, for example, bioavailability that is bioequivalent to the RLD, improved subject compliance (i.e., ability to easily take the right capsule during the correct period), reducing food and allergy effects due to administration, and reducing required prescribed dosage levels in order to achieve efficacy of the drug product.

In some embodiments, progesterone is fully or partially solubilized. The form of the API (i.e., being in solution), and other factors and conditions, may account for the increased bioavailabilty of progesterone as compared to the RLD.

In some embodiments, the pharmaceutical composition is delivered via a gelatin capsule delivery vehicle. In these embodiments, the pharmaceutical composition is a liquid pharmaceutical composition. Accordingly, the pharmaceutical composition of such embodiments is encapsulated in the gelatin capsule. The inclusion of the capsules in blister packs, as described elsewhere herein, ensures that subjects will receive the right dosage during the correct period of time.

In some embodiments, the gelatin capsules are softgels. Other forms of administration (i.e. injection, intra-muscular, etc.) can cause pain, discomfort, or irritation, especially when frequent administration is required. Softgels eliminate these problems, while minimizing adverse tastes. Softgels can be administered orally or can be administered locally. In some embodiments, the softgel is administered orally.

Through extensive experimentation, various medium chain fatty acid esters of glycerol and propylene glycol demonstrated one or more favorable characteristics for development as a human drug product. In one embodiment, the solubilizing agent was selected from at least one of a solvent or co-solvent. Suitable solvents and co-solvents include any mono-, di-, or triglyceride and glycols, and combinations thereof.

In other embodiments, the solubilizing agent was selected from one or more C6 to C12 fatty acid mono-, di-, or triesters of glycerol, e.g., one or more C6 to C14 triglycerides, e.g., one or more C6 to C12 triglycerides, such as one or more C8-C10 triglycerides. Thus, in certain embodiments, the pharmaceutical composition comprises progesterone that is at least about 75% solubilized in a solubilizing agent comprising one or more C6 to C14 medium chain fatty acid mono-, di-, or triglycericdes and, optionally, a thickening agent.

In still other embodiments, the pharmaceutical composition comprises progesterone that is at least about 75% solubilized one or more C6 to C12 medium chain fatty acid mono-, di-, or triglycerides, e.g., one or more C6 to C14 triglycerides, e.g., one or more C6 to C12 triglycerides, such as one or more C8-C10 triglycerides. These embodiments specifically contemplate the progesterone being at least 85% solubilized, at least 90% solubilized, at least 95% solubilized, and in certain instances, 100% solubilized. In other embodiments, estradiol or a combination of progesterone and estradiol is included in the pharmaceutical compositions as the one or more APIs.

As noted previously herein, liquid pharmaceutical compositions are preferably liquid at room temperature. Accordingly, gels, hard fats, or other solid forms that are not liquid at room or body temperature are less desirable in embodiments of the pharmaceutical composition that are liquid. In certain embodiments, where a non-ionic surfactant such as GELUCIRE or TEFOSE to increase viscosity, the non-ionic surfactant may be solid at room temperature. In those situations, the non-ionic surfactant may require melting to mix with one or more APIs solubilized in a fatty acid-glycol ester. In this embodiment, the resultant composition is advantageously liquid, not solid. However, in these embodiments, the resultant pharmaceutical composition remains liquid, albeit with greater viscosity, although it is still not a solid.

In other embodiments, the pharmaceutical composition comprises progesterone, a medium chain solubilizing agent, and a thickening agent as the only essential ingredients delivered via a softgel delivery vehicle. Non-essential ingredients, e.g., colorants, antioxidants, preservatives, or other excipients may be included as well. Other embodiments comprise one or more APIs.

Additional ingredients can be incorporated in amounts that do not materially change the solubility of the progesterone, the pharmacokinetics of the pharmaceutical composition, or the efficacy of the pharmaceutical composition. Other factors that should be considered when adjusting the ingredients of the pharmaceutical composition include taste, water regulation, and other relevant factors, for example those that would lead to reduced patient compliance.

In softgel embodiments, mucoadhesive agents, gelling agents, dispersing agents, or the like would not be included because of effects some of these ingredients may have on bioavailability of the API(s) in the digestive system.

Methods

Pharmaceutical compositions in different embodiments may be administered alone or combination with one or more other drugs (or as any combination thereof). For example, compositions in accordance with embodiments including one or more other drugs may also comprise estradiol. In such compositions, estradiol is also an API.

In certain embodiments, and as discussed elsewhere herein, the pharmaceutical composition disclosed herein can be administered orally in a softgel. As the softgel dissolves after administration, the inner volume may come into fluid communication with the digestive system such that the progesterone present in the pharmaceutical composition can be absorbed systemically. Oral administration may involve swallowing, so that the pharmaceutical composition enters the gastrointestinal tract. Alternatively, buccal or sublingual administration may be employed such that the pharmaceutical composition enters the bloodstream directly from the mouth.

In embodiments where hard shell capsules are employed, the method of administration is typically oral. Hard capsules or softgels may be arranged in blisters or cartridges or bottles.

In certain embodiments, a 28-day or monthly regimen of capsules can be packaged in a single kit (e.g., a blister pack) having delivery days identified to improve subject compliance. One or more of the capsules may contain no progesterone. A blister pack can have a plurality of scores or perforations separating blister pack into 28 days. Each day may further comprise a single blister or a plurality of blisters. In various embodiments, each dose (e.g., each softgel) may contain solubilized, partially solubilized, or partially suspended progesterone in any of the amounts previously set forth herein, though may, in certain instances, include 100, 150, or 200 mg of progesterone. In addition, kits having other configurations are also contemplated herein. For example, without limitation, kits having such blister packs may contain any number of capsules.

In additional embodiments, progesterone is formulated for intraperitoneal, percutaneous, subcutaneous, intra-muscular, and atomization administration (i.e. such as with nasal mist administration).

In still other embodiments, the pharmaceutical compositions are administered according to other techniques known to those skilled in the art, which may include, but are not limited to: tablets, film-coated tablets, prolonged-release tablets, modified-released tablets, effervescent tablets, orodispersible tablets, sachets, dry powders used to form suspension; or liquid dosage forms.

Compositions in accordance with the various embodiments disclosed herein may be used to treat or prevent endometrial hyperplasia, prevent secondary amenorrhea, or mitigate or treat the effects of estradiol supplementation. In certain embodiments, compositions comprising progesterone may be co-administered with estradiol or co-formulated with estradiol.

In other embodiments, formulations in accordance with various embodiments may be used to treat or prevent preterm delivery in pregnant women, including in certain women having a shortened cervix. In various embodiments, a capsule, for example a softgel capsule, may be opened and the fill material applied in or around the vagina. However, in various embodiments the capsules are taken orally.

In still further embodiments, formulations in accordance with various embodiments may be used to treat menopause-related symptoms, including vasomotor symptoms, for example, in relation to treatment of hypoestrogenism related symptoms including hot flashes and night sweats (vasomotor symptoms), sleep disturbances, mood changes, vulvo-vaginal atrophy; and osteoporosis and endometrial hyperplasia reduction.

In still further embodiments, formulation in accordance with various embodiments may be used to treat amenorrhea.

Additional objects of this disclosure include: providing increased patient compliance secondary to ease of use; providing increased physician adoption secondary to ease of use/instruction with less worry of side effects from inappropriate usage; providing decreased side-effects from erroneous use (decreased irregular bleeding); providing better efficacy/control of symptoms secondary to appropriate use; reducing the metabolic and vascular side effects of the commonly used synthetic progestins when administered alone or in combination with an estrogen (norethindrone acetate, medroxyprogesterone acetate, etc.) including, for example, stroke, heart attacks, blood clots and breast cancer.

Enhanced Bioavailability

In certain embodiments, the formulations disclosed herein provide enhanced bioavailability of progesterone when compared to conventional progesterone formulations. As a result of this improved bioavailability, certain embodiments of the formulations disclosed herein allow for a reduction in the quantity of progesterone administered to a person in need thereof while still providing the providing the benefits of a dosage form containing the greater amount of progesterone.

As such, and in certain embodiments, a formulation of this disclosure can include less than 200 mg of progesterone while still having an acceptable PK profile. In particular, embodiments, the formulation can include about 175 mg of progesterone, about 170 mg of progesterone, about 165 mg of progesterone, about 160 mg of progesterone, about 159 mg of progesterone, about 158 mg of progesterone, about 157 mg of progesterone, about 156 mg of progesterone, about 155 mg of progesterone, about 154 mg of progesterone, about 153 mg of progesterone, about 152 mg of progesterone, about 151 mg of progesterone, about 150 mg of progesterone, about 149 mg of progesterone, about 148 mg of progesterone, about 147 mg of progesterone, about 146 mg of progesterone, about 145 mg of progesterone, about 170 mg of progesterone, about 140 mg of progesterone, about 135 mg of progesterone, about 170 mg of progesterone, about 130 mg of progesterone, about 125 mg of progesterone, about 120 mg of progesterone, about 115 mg of progesterone, about 110 mg of progesterone, about 105 mg of progesterone, or about 100 mg of progesterone. In still further embodiments, the formulation can have exactly the amounts of progesterone noted above, e.g. exactly 175 mg of progesterone, exactly 170 mg of progesterone, etc.

In certain embodiments, this disclosure provides a formulation including less than 200 mg of progesterone having an AUC_{0-∞} in (ng/ml)*hr of from about 5 to about 500, from about 5 to about 400, from about 5 to about 300, from about 5 to about 270, from about 20 to about 200, from about 25 to about 150, or from about 25 to about 140. In particular embodiments, the formulation including less than 200 mg progesterone can have an AUC_{0-∞} of about 137 (ng/ml)*hr±95%. In particular embodiments, the formulation can have about 150 or exactly 150 mg progesterone.

In certain embodiments, this disclosure provides a formulation including less than 200 mg of progesterone having an AUC₀₋ₜ in (ng/ml)*hr of from about 5 to about 500, from about 5 to about 400, from about 5 to about 300, from about 5 to about 240, from about 20 to about 200, from about 25 to about 150, or from about 25 to about 140. In particular embodiments, the formulation including less than 200 mg progesterone can have an AUC₀₋ₜ of about 120 (ng/ml)*hr±95%. In particular embodiments, the formulation can have about 150 or exactly 150 mg progesterone.

In certain embodiments, this disclosure provides a formulation including less than 200 mg of progesterone having a Cₘₐₓ in ng/ml of from about 3 to about 350, from about 3 to about 325, from about 3 to about 300, from about 3 to about 250, from about 3 to about 240, and from about 3 to about 230. In particular embodiments, the formulation including less than 200 mg progesterone can have a Cₘₐₓ of about 75 ng/ml±95%. In particular embodiments, the formulation can have about 150 or exactly 150 mg progesterone.

Although the amount of progesterone is typically less than 200 mg, in certain embodiments, the amount of progesterone can be about 300 mg. In such embodiments, the formulation can have the following PK parameters upon administration:

In certain embodiments, this disclosure provides a formulation including about 300 mg of progesterone having an AUC_{0-∞} in (ng/ml)*hr of from about 10 to about 1000, from about 10 to about 800, from about 10 to about 600, from about 10 to about 540, from about 40 to about 400, from about 50 to about 300, or from about 50 to about 280. In particular embodiments, the formulation including about 300 mg progesterone can have an AUC_{0-∞} of about 274 (ng/ml)*hr±95%.

In certain embodiments, this disclosure provides a formulation including about 300 mg of progesterone having an AUC₀₋ₜ in (ng/ml)*hr of from about 10 to about 1000, from about 10 to about 800, from about 10 to about 600, from about 10 to about 480, from about 40 to about 400, from about 50 to about 300, or from about 50 to about 280. In particular embodiments, the formulation including about 300 mg progesterone can have an AUC₀₋ₜ of about 240 (ng/ml)*hr±95%.

In certain embodiments, this disclosure provides a formulation including about 300 mg of progesterone having a Cₘₐₓ in ng/ml of from about 6 to about 700, from about 6 to about 650, from about 6 to about 600, from about 6 to about 500, from about 6 to about 480, and from about 6 to about 460. In particular embodiments, the formulation including about 300 mg progesterone can have a Cₘₐₓ of about 150 ng/ml±95%.

Bioavailability comparisons to commercially available forms, such as tablet forms, may be determined by standard pharmacokinetic techniques

In accordance with various embodiments, food effects are reduced, e.g., relative to comparative progesterone products.

In accordance with various embodiments, formulations do not include peanut oil. The lack of peanut oil obviates the risk posed to those having peanut-based allergies.

Measurement of Efficacy

Efficacy can be measured using standard techniques known in the art. However in certain embodiments, subjects are administered progesterone. After administration of the progesterone, endometrial biopsies can be performed by a board-certified gynecologist. Procedures, instruments used, and observations are documented in the subject's file.

The resulting biopsy specimens can then processed by a central laboratory. The central laboratory includes a chartered pathology committee of independent pathologists who are experts in the field of endometrial pathology to assess all endometrial biopsy sample.

In certain embodiments, treatment with the pharmaceutical compositions described herein resulted in complete and partial secretory activity. In cases of complete secretory activity, subjects experienced 1) glands with secretory changes, and 2) stromal predecidual changes. In cases of partial secretory activity, subjects experienced 1) glands with secretory changes, or 2) stromal predecidual changes.

In certain embodiments, subjects are administered pharmaceutical compositions as described herein, while other subjects are administered placebos. Exemplary test scenarios are described in the Example section, below. In these embodiments, secretory activity is measured as a proportion of subjects at Cycle 3 Day 24±1 day on active treatment (200 mg progesterone/day, 225 mg progesterone/day, or 300 mg progesterone/day) compared to placebo with complete secretory activity on endometrial biopsy (referenced in the examples as the “primary efficacy endpoint”).

In these embodiments, secretory activity is also measured as a proportion of subjects at Cycle 3 Day 24±1 day on active treatment (200 mg progesterone/day, 225 mg progesterone/day, or 300 mg progesterone/day) compared to placebo with total secretory activity (defined as the aggregate of partial and complete secretory activity) on endometrial biopsy. Included in this measurement is an observation of the proportion of subjects reporting withdrawal bleeding at cycle 2 on or after cycle day 21 or within 7 days (including 7th day) after completion of blinded treatment at cycle 2 (this and the secretory measurement of the preceding sentence are referenced in the examples as the “,secondary efficacy endpoints”).

Statistical Measurements

Pharmacokinetics of the pharmaceutical composition disclosed herein can be calculated using statistical analyses. In particular embodiments, Analysis of Variance (“ANOVA”) or Analysis of CoVariance (“ANCOVA”) are used to evaluate differences between a subject receiving treatment with a pharmaceutical composition comprising an active pharmaceutical composition (for example, a pharmaceutical composition comprising progesterone) and a subject receiving treatment with a placebo (for example, the same pharmaceutical composition but without progesterone) or a reference drug. A person of ordinary skill in the art will understand how to perform statistical analysis of the data collected.

Among the data collected or calculated are PK parameters for pharmacokinetic evaluation and analysis, including, but not limited to, AUC, Cₘₐₓ, and Tₘₐₓ. The pharmacokinetic evaluation was carried out by a research lab using statistical and analytical software, which could include, but is not limited to. WinNonlin® software (version 5.3), and using SAS version 9.2.

### Specific Embodiments

Through extensive trial-and-error testing of various fatty acid esters of glycerol and other glycols, embodiments of the invention have been invented that have one or more favorable characteristics for development as a human drug product. Such favorable characteristics include those described above, e.g., improved PK properties and reduced inter- and intra-patient variability.

Such embodiments include an encapsulated liquid pharmaceutical formulation for orally administering progesterone to a mammal in need thereof, said formulation comprising: progesterone, as the sole active pharmaceutical ingredient, in ultra-micronized form suspended in a carrier that comprises a medium chain fatty acid-glycol ester or mixtures thereof and a non-ionic surfactant comprising a polyethylene glycol fatty acid ester.

In particular embodiments, the progesterone can be ultramicronized.

In certain embodiments, the progesterone is suspended or solubilized in one or more solubilizing agents such as one or more C6 to C14 fatty acid mono-, di-, or triesters of glycerol, including, but not limited to, one or more C6 to C14 triglycerides, one or more C6 to C12 triglycerides, or one or more C8-C10 triglycerides, as well as combinations thereof. An example of a solubilizing agent that provides beneficial properties is MIGLYOL, and in particular MIGLYOL 812.

In such general and more specific embodiments, the non-ionic surfactant is a polyethylene glycol saturated or unsaturated fatty acid ester or diester. In certain such embodiments, the non-ionic surfactant comprises C8 to C18 fatty acid esters of glycerol and polyethylene glycol. An example of a non-ionic surfactant that provides beneficial properties is GELUCIRE, e.g., GELUCIRE 44/14.

In certain such embodiments, the non-ionic surfactant has a HLB value of about 15. An illustrative example of such surfactant is GELUCIRE 44/14.

### EXAMPLES

The formulations and methods described herein are now further detailed with reference to the following examples. These examples are provided for the purpose of illustration only and the formulations and methods described herein should in no way be construed as being limited to these examples. Rather, the formulations disclosed herein should be construed to encompass any and all variations which become evident as a result of the teaching provided herein.

### Example 1

In an exemplary embodiment, a capsule is provided containing a fill material comprising a formulation set forth in one of Tables 2, 2A, or 2B

TABLE 2

| Ingredient | mg/Capsule | % | Function |
|---|---|---|---|
| Ultra-micronized | 200.00 | 30.77 | Active |
| Progesterone | | | |
| Medium Chain | qs | qs | Solubilizing Agent |
| Triglyceride | | | |
| (MIGLYOL 812 or | | | |
| equivalent) | | | |
| Lecithin Liquid | 1.63 | 0.25 | Lubricant/Emulsifier |
| Butylated | 0.13 | 0.02 | Antioxidant |
| Hydroxytoluene (also | | | |
| referred to as “BHT”) | | | |

TABLE 2A

| Ingredient | mg/Capsule | % | Function |
|---|---|---|---|
| Progesterone | 150 | 33.3 | Active |
| Medium | 292.3 | 65.0 | Solubilizing Agent |
| Chain Triglyceride | | | |
| (MIGLYOL 812 | | | |
| or equivalent) | | | |
| Lauroyl | 7.7 | 1.7 | Lubricant/Emulsifier |
| polyoxyl-32- | | | |
| glycerides | | | |
| (GELUCIRE | | | |
| 44/14 or equivalent) | | | |

TABLE 2B

| Ingredient | mg/Capsule | % | Function |
|---|---|---|---|
| Progesterone | 75 | 33.3 | Active |
| Medium Chain | 146.2 | 65.0 | Solubilizing Agent |
| Triglyceride | | | |
| (MIGLYOL 812 | | | |
| or equivalent) | | | |
| Lauroyl | 3.8 | 1.7 | Lubricant/Emulsifier |
| polyoxyl-32- | | | |
| glycerides | | | |
| (GELUCIRE | | | |
| 44/14 or equivalent) | | | |

The formulation in Table 2 is prepared as follows: MIGLYOL is heated to about 45° C. GELUCIRE 44/14 is added and mixed until dissolved. BHT is added and mixed until dissolved. Progesterone is suspended and passed through a colloid mill. The resultant fill mass can be used for encapsulation.

The formulations in Tables 2A and 2B are prepared as follows: melt Gelucire 44/14 by heating it to about 45-50° C.: once Gelucire 44/14 is completely melted, add MIGYOL 812 and mix/stir until dissolved; continue mixing/stirring; during the mixing/stirring, slowly add progesterone to the solution; and, after all progesterone has been added, continue mixing for a period of time to ensure proper suspension and near dissolution equilibrium. The suspended progesterone is then passed through a colloid mill. De-gassing and applying a vacuum for complete de-aeration of the fill mass is conducted. The resultant fill mass can be used for encapsulation.

### Example 2

In an exemplary embodiment, a capsule is provided containing a fill material comprising:

TABLE 3

| Ingredient | % | mg/Capsule | Function |
|---|---|---|---|
| Ultra-micronized | 30.77 | 200.00 | Active |
| Progesterone | | | |
| Medium Chain | 65.93 | 428.55 | Solubilizing Agent |
| Triglyceride | | | |
| (MIGLYOL 812 or | | | |
| equivalent) | | | |
| Lauroyl polyoxyl-32- | 3.00 | 19.50 | Suspending Agent |
| glycerides | | | |
| (GELUCIRE 44/14 or | | | |
| equivalent) | | | |
| Butylated | 0.03 | 1.95 | Antioxidant |
| Hydroxytoluene | | | |
| Total | 100 | 650 | |

In various embodiments, amounts of MIGLYOL may be present in a range from about 35-95% by weight; GELUCIRE 44/14 from about 0.5-30% by weight; and BHT from about 0.01-0.1% by weight.

### Example 3

### Progesterone Solubility

In various embodiments, both estradiol and progesterone may be independently dissolved in a solubilizing agent. In various embodiments, the solubility of both estradiol and progesterone will be such that a therapeutically effective dose may be obtained in a reasonably sized mass, generally considered to be between 1 mg and 1200 mg, preferably suitable for encapsulation in a size 3 to 22 oval or oblong capsule. For example, in various embodiments, 50 mg to 100 mg of progesterone may be dissolved in a volume of solubilizing agent; i.e., the solubility would be 50 mg to 100 mg per capsule.

MIGLYOL was attempted, and while it can be considered a good carrier for progesterone, it alone did not provide a desirable level of solubilization of estradiol (e.g., solubility of 12 mg/g may be desirable in various embodiments). Thus, MIGLYOL, including without limitation MIGLYOL 812, may be used in embodiments comprising fully solubilized, partially solubilized, and suspended progesterone.

As can be seen in Table 4, the solubility of progesterone in CAPMUL MCM is ˜73 mg/g. Therefore, by suspending 200 mg progesterone in 400 mg of solvent, part of the dose (˜14%) is already dissolved and the remaining is still a suspension. In some aspects and embodiments, it is desired to minimize the partial solubility of progesterone in the formulation in order to minimize the possibility of recrystallization. Based on 73 mg/g solubility, the capsule size required to make a capsule of 50 mg solubilized progesterone would be 685 mg. Based on 95 mg/g solubility, a 50 mg progesterone capsule would require a 526 capsule size. The other capsule sizes required based on each respective solubility below includes: 1,799 mg, 579 mg, 709 mg, and 871 mg. Capsule size amounts based on respective solubilities will generally be at least 10% greater than the calculated value in order to ensure the progesterone remains in solution. Thus, a 50 mg progesterone capsule based on 73 mg/g solubility would require a 685 mg capsule, and with the at least 10% addition, it would require approximately a 754 mg sized capsule. Based on each respective solubility listed below, the capsule sizes include (approximately): 579 mg, 1979 mg, 637 mg, 780 mg, and 958 mg respectively. These values, and their corresponding 10% additions are shown in Table 4.

TABLE 4

| | Progesterone |
|---|---|
| Ingredient | Solubility (mg/g) |
| CAPMUL MCM | 73.4 |
| CAPMUL PG8 | 95 |
| MIGLYOL 812 | 27.8 |
| CAPMUL MCM:GELUCIRE 44/14 (9:1) | 86.4 |
| CAPMUL MCM:GELUCIRE 44/14 (7:3) | 70.5 |
| CAPMUL MCM:GELUCIRE 44/14 (6:3) | 57.4 |

In addition, it has been found that the solubility of progesterone in a solvent of CAPMUL MCM in combination with GELUCIRE 44/14 in a 9:1 ratio increases the solubility to approximately 86 mg/g. Therefore, in various embodiments, progesterone or estradiol may be dissolved in a CAPMUL MCM and GELUCIRE 44/14 system, wherein the ratio of CAPMUL MCM to GELUCIRE 44/14 is 9:1.

TABLE 5

| | Progesterone |
|---|---|
| Ingredient | Solubility (mg/g) |
| CAPMUL MCM:GELUCIRE 44/14 (9:1) | 86.4 |
| CAPMUL MCM:GELUCIRE 44/14 (7:3) | 70.5 |
| CAPMUL MCM:GELUCIRE 44/14 (6:4) | 57.4 |

### Example 4

In an exemplary embodiment, a capsule is provided containing a fill material having suspended progesterone comprising:

TABLE 6

| Ingredient | mg/Capsule | % | Function |
|---|---|---|---|
| Micronized | 200.00 | 30.77 | Active |
| Progesterone | | | |
| Medium Chain | qs | qs | Solubilizing Agent |
| Triglyceride | | | |
| (MIGLYOL 812 or | | | |
| equivalent) | | | |
| Lecithin Liquid | 1.63 | 0.25 | Lubricant/Emulsifier |
| Butylated | 0.13 | 0.02 | Antioxidant |
| Hydroxytoluene (also | | | |
| referred to as “BHT”) | | | |

The above formulation is prepared as follows: MIGLYOL is heated to about 45° C. GELUCIRE 44/14 is added and mixed until dissolved. BHT is added and mixed until dissolved. Progesterone is suspended and passed through a colloid mill. The resultant fill mass can be used for encapsulation.

In an exemplary embodiment, a capsule is provided containing a fill material having partially solubilized progesterone comprising:

TABLE 7

| | Qty/Capsule | | Qty/Capsule | Amount/ |
|---|---|---|---|---|
| Ingredient | (mg) | % w/w | (mg) | Batch (kg) |
| Micronized | 200.00 | 33.33 | Active | 2.0 |
| Progesterone, | | | | |
| USP | | | | |
| Monoglycerides/ | 394.0 | 65.67 | Solubilizing | 3.94 |
| diglycerides/ | | | Agent | |
| triglycerides of | | | | |
| caprylic/capric acid | | | | |
| (CAPMUL MCM) | | | | |
| Lauroyl polyoxyl-32- | 6.0 | 1 | Lubricant/ | 0.06 |
| glycerides | | | Emulsifier | |
| (GELUCIRE 44/14 | | | | |
| or equivalent) | | | | |
| Total | 600.00 mg | 100 | | 6.0 kg |

For suspensions of progesterone and partially solubilized progesterone, GELUCIRE 44/14 may be added at 1% to 2% w/w to increase viscosity. The above formulation is prepared as follows: CAPMUL MCM is heated to about 65° C. GELUCIRE 44/14 is added and mixed until dissolved. Heat is removed. Progesterone is added and the mixture is passed through a colloid mill. The resultant fill mass can be used for encapsulation.

### Example 5

In particular embodiments, a capsule is provided containing a pharmaceutical composition having fully solubilized, partially solubilized, or suspended progesterone comprising the components according to the formulations specified in Tables 8 and 9:

TABLE 8

| Ingredient | % | mg/Capsule | Function |
|---|---|---|---|
| Micronized | 30.77 | 200.00 | Active |
| Progesterone | | | |
| Medium Chain | 65.93 | 428.55 | Carrier |
| Triglyceride | | | |
| (MIGLYOL 812 or | | | |
| equivalent) | | | |
| Lauroyl polyoxyl-32- | 3.00 | 19.50 | Suspending Agent |
| glycerides | | | |
| (GELUCIRE 44/14 or | | | |
| equivalent) | | | |
| Butylated | 0.03 | 1.95 | Antioxidant |
| Hydroxytoluene | | | |
| Total | 100 | 650 | |

TABLE 9

| Ingredient | mg/Capsule | % | Function |
|---|---|---|---|
| Progesterone | 200.00 | 33.33 | Active |
| Medium Chain | 389.60 | 64.93 | Solubilizing |
| Triglyceride | | | Agent |
| (MIGLYOL 812 or | | | |
| equivalent) | | | |
| Lauroyl polyoxyl-32- | 10.00 | 1.67 | Non-ionic |
| glycerides (GELUCIRE | | | Surfactant |
| 44/14 or equivalent) | | | (suspending |
| | | | agent) |
| Butylated | 0.40 | 0.07 | Antioxidant |
| Hydroxytoluene | | | |
| Total | 600.00 | 100.0 | |

The pharmaceutical composition above can be prepared in accordance with the procedures noted in prior examples.

### Example 6

A gel mass can be prepared in order to encapsulate the pharmaceutical compositions of the various Examples herein.

Gel mass compositions were formulated and produced according to the following steps. Purified water (22.2 kg) and glycerin (10.8 kg) were charged into a stainless steel tank with mixing and heated to a temperature of 80±5° C. Hydrolyzed gelatin (1.8 kg) and gelatin 200 bloom limed bone, NF (24.0 kg) were then added to the water/glycerin mixture and were mixed until all solids were completely dissolved. This resulted in the formation of a gel mass. The resulting gel mass was de-gassed under vacuum. Coloring agents OPATINT® white (0.6 kg) and OPATINT® red (0.6 kg) were then added to the gel mass and the resultant was mixed for about 5 minutes. The resultant was then de-gassed under vacuum for a sufficient period of time and ultimately passed to an encapsulation device for preparation of gel capsules of the types disclosed herein.

### Example 7

### Bioavailability Assessment—Fasted

A randomized single-dose oral bioequivalence study comparing 200 mg ultra-micronized progesterone capsule test product (T) and 200 mg PROMETRIUM® (progesterone) capsules (Abbott Laboratories, Abbott Park, Ill.) reference product (R) is conducted. Subjects are administered a single 200 mg dose of either test product (T) or the reference product (R) under fasting conditions, for example, subjects fasted at least 10.0 hours prior to dosing. Blood is collected pre-dose and post-dose. Pre-dose samples are collected at approximately −01.00, −00.50, and 00.00 hours. Post-dose samples are collected at approximately 01.00, 02.00, 03.00, 04.00, 05.00, 06.00, 07.00, 08.00, 09.00, 10.00, 12.00, 18.00, 24.00, 36.00 and 48.00 hours. Standard meals are provided at 04.00, 09.00, 13.00, 25.00, 29.00, 33.00 and 37.00 hours post-dose.

Pharmacokinetic measurements are assessed including Cmax, AUC and optionally Tmax. Comparative bioavailability of the test product (T) and reference product are assessed.

### Example 8

### Bioavailability Assessment—Fed

The procedures for determining bioavailability under fasted conditions are repeated except that subjects are administered a single 200 mg dose of either test product (T) or reference product (R) immediately following a high fat meal, for example, within 30 minutes of dosing. Blood is collected pre-dose and post-dose. Pre-dose samples are collected at approximately −01.00, −00.50, and 00.00 hours. Post-dose samples are collected at approximately 01.00, 02.00, 03.00, 04.00, 05.00, 06.00, 07.00, 08.00, 09.00, 10.00, 12.00, 18.00, 24.00, 36.00 and 48.00 hours. Standard meals are provided at 04.00, 09.00, 13.00, 25.00, 29.00, 33.00 and 37.00 hours post-dose. Pharmacokinetic measurements are assessed including Cₘₐₓ, AUC and optionally Tₘₐₓ. Bioavailability of the test product (T) in reference to the reference product is assessed. The effect of food on the comparative bioavailability of the test product (T) and the reference product (R) are also assessed.

### Example 9

Method of manufacture in accordance with various embodiments are shown in FIGS. 1-3. With reference to FIG. 1, method of fill material, i.e. fill mass, preparation 100 is shown. Operation 102 comprises mixing a carrier, a lubricant, and an antioxidant as described herein. For example, lecithin and butylated hydroxytoluene may be mixed with one or more medium chain mono-, di- or triglycerides, or combinations thereof. Mixing may be facilitated by an impellor, agitator, or other suitable means. Operation 102 may be performed under an inert or relatively inert gas atmosphere, such as nitrogen gas N2. Mixing may be performed in any suitable vessel, such as a stainless steel vessel.

Operation 104 may comprise mixing ultra-micronized progesterone into the mixture of the carrier, the lubricant, and the antioxidant. A pasty substance is thus formed. Mixing may occur in a steel tank or vat. Mixing may be facilitated by an impellor, agitator, or other suitable means. Operation 104 may be performed under an inert or relatively inert gas atmosphere, such as nitrogen gas N2. Operation 106 comprises degasing. The resulting mixture from operation 106 may comprise a fill material suitable for production into a softgel capsule.

With reference to FIG. 2, softgel capsule, i.e. gel mass, production 200 is shown. Operation 202 comprises mixing glyercin with water. The water used in operation 202 may be purified by any suitable means, such as reverse osmosis, ozonation, filtration (e.g., through a carbon column) or the like. Mixing may be facilitated by an impellor, agitator, or other suitable means. Operation 202 may be performed under an inert or relatively inert gas atmosphere, such as nitrogen gas N₂. Heating may be performed until the temperature reaches 80±5 C.

Operation 204 comprises the addition of gelatin to the glycerin water mixture. Mixing may be facilitated by an impellor, agitator, or other suitable means. Operation 204 may be performed under an inert or relatively inert gas atmosphere, such as nitrogen gas N₂. A vacuum may be drawn in operation 204 to de-aerate.

Operation 206 comprises addition of a coloring agent such as a dye. A coloring agent may comprise products sold under the trademark OPATINT or other suitable agent. Operation 206 may be performed under an inert or relatively inert gas atmosphere, such as nitrogen gas N₂. Operation 208 comprises degasing. The resulting mixture from operation 208 may comprise a gel capsule material suitable for use as a gel capsule in production of a softgel capsule.

With reference to FIG. 3, softgel capsule assembly process 300 is shown. Operation 302 comprises heating the fill material. The fill material may be heated to any suitable temperature. In various embodiments, the fill material is heated to 30° C.+/−3° C. Fill material maybe heated in a fill hopper. A fill hopper may comprise a device configured to hold a volume of the fill material or to dispense the fill material in controlled volumes.

Operation 304 comprises filling a gel mass. A gel mass may be taken from the gel capsule material produced in operation 208 of FIG. 2. Filling may be performed by injecting, placing, or otherwise disposing the fill material within a volume defined by the gel capsule material. The filling may occur in an encapsulator. The spreader boxes may be a temperature of 55° C.+/−10° C. The wedge temperature may be 38° C.+/−3° C. The drum cooling temperature may be 4° C.+/−2° C. The encapsulator may be lubricated using MIGLYOL 812. Operation 304 thus produces one or more softgel capsules. Filling may comprise producing a ribbon of thickness 0.85±0.05 mm using spreader box knobs. The fill material may be injected into the gel to produce a fill weight having target weight ±5% (i.e., 650±33 mg and 325±16.3 mg).

Operation 306 comprises drying the softgel capsules. Drying may be performed in a tumble dryer, tray dryer, or combinations thereof. For example, drying may be performed in a tumble drying basket for between about 10 minutes and about 120 minutes. Drying may continue in a drying room for about 24 hours to about 72 hours. Polishing may be performed with isopropyl alcohol.

### Example 10

### Stability Study

In accordance with various embodiments, formulations in accordance with various embodiments have an exemplary shelf life of 3 months with storage at 25±2° C./60±5% RH in 75 cc HDPE white, opaque bottles with a 38/400 mm white child resistant cap.

Packaging during testing comprises a 75 cc round HDPE bottle and 33 mm cap. A Brasken FPT 300F resin is associated with the cap. Testing criteria include visual appearance, assay of progesterone, dissolution, content uniformity and microbial limits testing.

Three test groups are created. Test group 1 comprises a test at 40° C./75% RH. Test group 2 comprises a test at 30° C./65% RH. Test group 3 comprises a test at 25° C./60% RH. Test group 1 is tested for visual appearance, assay of ultra-micronized progesterone, and dissolution at months 1, 2, 3, and 6. Test group 2 is tested for visual appearance, assay of ultra-micronized progesterone, and dissolution at months 0, 1, 2, 3, 6, and 12. Test group 3 is tested for visual appearance, assay of ultra-micronized progesterone, and dissolution at months 0, 1, 2, 3, 6, 12 and 24.

### Example 11

A particle size analysis is conducted by using a Beckman Coulter LS 13 320 Laser Diffraction Particle Size Analyzer (the “Beckman Device”). The Beckman Device uses laser diffraction to determine particle size. A sample of a formulation in accordance with various embodiments is provided. The Beckman Device particle sensor yields that the sample has an X50 of 6.67 μm, an X75 of 14.78 μm, and an X25 of 2.193 μm.

### Example 12

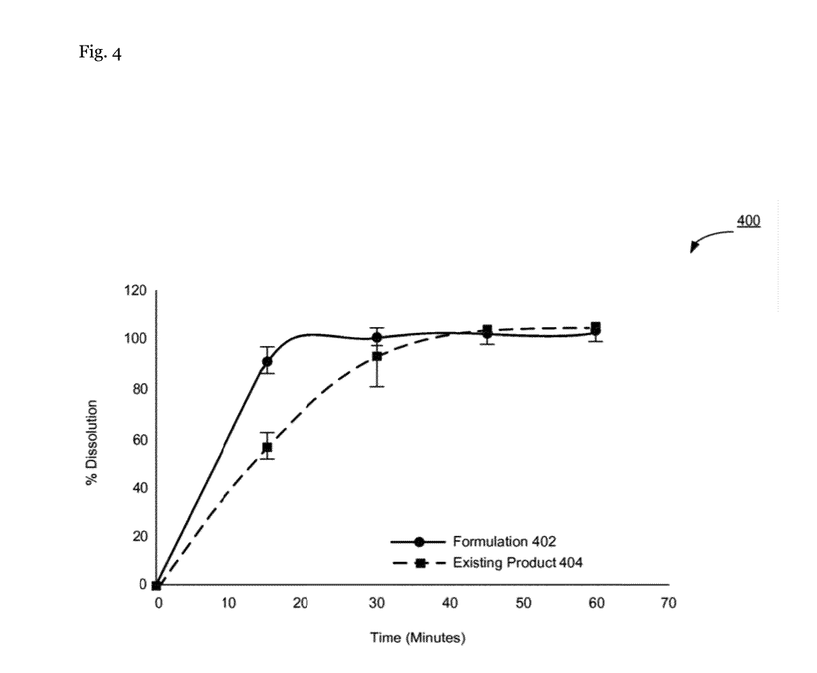

A dissolution study was performed using a formulation in accordance with various embodiments. The results of the dissolution study are shown in FIG. 4.

The dissolution study was performed using a United States Pharmacopoeia dissolution apparatus 3 (reciprocating cylinder) (“USP Apparatus 3”). The USP Apparatus 3 was set to 30 dips per minute. Two hundred fifty mL (250 mL) of a solution of. 1N HCL with 3% sodium lauryl sulfate was used at 37° C.

FIG. 4 shows dissolution percentage in the y axis over time in minutes on the x axis. A formulation in accordance with various embodiments is shown having circular dots, and is labeled formulation 402. An existing commercial pharmaceutical product containing progesterone is shown having square dots and is labeled existing product 404. As shown in FIG. 4, formulation 402 reaches a higher level of dissolution in a shorter time than existing product 404.

### Example 13

For the purposes of this Example, a particle size analysis is conducted by using the Beckman Device. A sample API comprising ultra-micronized progesterone in accordance with various embodiments is provided for analysis.

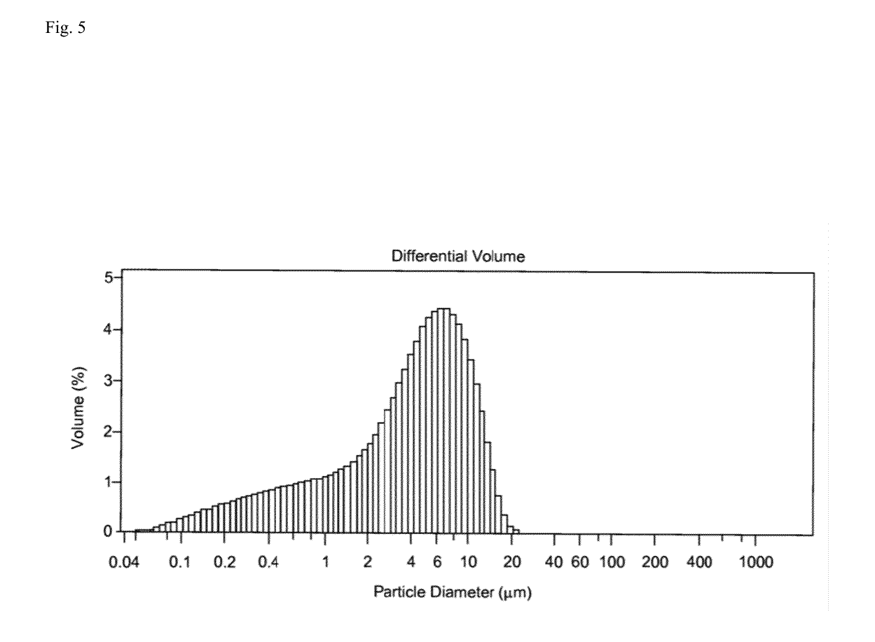

Approximately 0.01 g of a sample API in accordance with various embodiments was combined with Coulter 1B and 10 mL of deionized water. Sonication was performed for 15 seconds. The Beckman Device, equipped with a ULM, performed analysis for 90 seconds. The Beckman Device was configured to use the Fraunhofer optical model. The Beckman Device yielded that the sample has an X50 of 4.279 μm, an X75 of 7.442 μm, and an X25 of 1.590 μm. The Beckman Device also yielded that the mean particle size is 4.975 μm, the median particle size is 4.279 μm, the mode particle size is 6.453 μm, and the standard deviation is 3.956 μm. A graph of the particle distribution obtained is shown in FIG. 5.

### Example 14

### Dissolution

Dissolution studies were performed using a formulation of this invention comparing the dissolution of progesterone to the dissolution of PROMETRIUM and comparing the dissolution of estradiol to the dissolution of Estrace. In one study, a formulation of the invention in capsules comprising 200 mg of progesterone and 2 mg estradiol was used. In a second study, a formulation of the invention in capsules comprising 50 mg of progesterone and 2 mg estradiol was used.

The dissolution study was performed using a USP dissolution apparatus (reciprocating cylinder) (“USP Apparatus 3”). The apparatus was set to 30 dips per minute. 250 mL of a solution of 0.1N HCl with 3% sodium lauryl sulfate was used at 37 C.

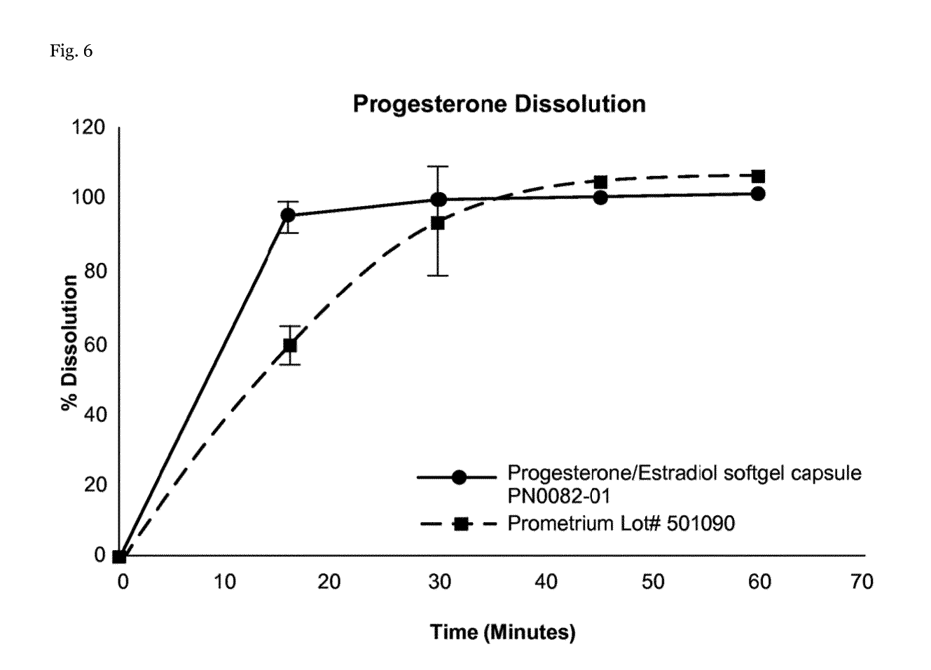

In both studies, progesterone was dissolved faster, and with smaller standard deviations, from the capsules of the invention than from PROMETRIUM. Dissolution of estradiol was comparable but marginally slower from the capsules of the invention than from Estrace. For illustrative purposes, a graph showing progesterone dissolution from the 200 mg progesterone capsule of the invention and from PROMETRIUM is attached as FIG. 6.

Both capsules of the invention were stable on storage in white HDPE bottles. Positive stability data were obtained with the 200 mg progesterone formulation over 6 months (>6 months data unavailable) and with the 50 mg progesterone formulation over 3 months (>3 months data unavailable).

### Example 15

### Bioavailability & Bioequivalence Assessment

This study was conducted to determine bioavailability and bioequivalence of reference product PROMETRIUM “R” (200 mg progesterone) and test product “T” as described in Table 9 herein. T was administered as a softgel capsule.

The study was an open-label, balanced, randomized, single-dose, two-treatment, three-period, three-sequence, crossover, partial replicate, reference-scaled oral bioequivalence study. A total of 72 healthy, adult, human, postmenopausal female subjects were enrolled in the study. Each subject was randomly assigned to a sequence (TRR, RTR, or RRT) such that each subject received T once and R twice during the course of the 32 day study (14 day washout between doses). R was administered twice so that the within subject variance could be calculated for later assessment of bioequivalence of the T and R formulations.

On study days 1, 15, and 29, patients who had been fasting for 10 hours were administered a high fat meal. 30 minutes after the meal, each patient was given a single softgel dose of T or, alternatively, R, in accordance with the patients' randomly assigned sequence. The dosage forms were taken with 240 ml of water. Subjects were housed in a clinical facility for at least 11 hours prior to dosing to at least 24 hours post dose.

A total of 20 (3×8 mL pre-dose and 17×6 mL post dose) blood samples were collected per subject after each dose. Pre-dose samples were collected at −1.00, −0.50, 0 hrs. Post dose samples were collected at 0.25, 0.50, 0.67, 0.83, 1.00, 1.33, 1.67, 2.00, 02.50, 3.00, 4.00, 6.00, 8.00, 12.00 24.00, 36.00 and 48.00 hours after dosing in vacutainers containing K₂EDTA. Based on an analysis of the collected blood samples, pharmacokinetic parameters including Cₘₐₓ, AUC₀₋ₜ, AUC_{0-∞}, and Tₘₐₓ were calculated using WinNonlin® version 5.3 (Pharsight Corporation). Although 72 patients were enrolled in the study, only data from the 62 patients who finished the study was used to calculate the values shown in Table 11, below.

TABLE 11

| | Mean Parameters (+/−SD) | | | |
|---|---|---|---|---|
| Treatments | | | Tₘₐₓ | AUC₀₋ₜ |
| (Dose Dosage | Cₘₐₓ | | (hr) | (ng/mL)*hr |
| form, route) | (ng/mL) | | Median | Mean |
| [Product ID] | Mean (% CV) | | (Range) | (% CV) |
| Test product | T | 102.5744 ± 139.2924 | 03.00 | 145.9243 ± 166.3317 |
| Progesterone | | | (0.83-08.00) | |
| Soft gel Capsule | | | | |
| 200 mg, | | | | |
| (Single dose) | | | | |
| Oral | | | | |
| Reference | R₁ | 83.8777 ± 142.4315 | 4.00 | 139.8621 ± 195.2669 |
| product | | | (01.00-12.00) | |
| PROMETRIUM ® | R₂ | 61.7121 ± 97.1097 | 4.00 | 98.6441 ± 130.9716 |
| (Progesterone) | | | (01.00-12.00) | |
| soft gel Capsule | | | | |
| 200 mg | | | | |
| (Single dose - 2 × | | | | |
| 200 mg), | | | | |
| Oral | | | | |

| | Mean Parameters (+/−SD) | | |
|---|---|---|---|
| Treatments | AUC_{0-∞} | | Kₑₗ |
| (Dose Dosage | (ng/mL) * hr | t_{1/2} (hr) | (hrs)⁻¹ |
| form, route) | Mean | Median | Mean |
| [Product ID] | (% CV) | (Range) | (% CV) |
| Test product | 169.2228 ± 172.1370 | 3.9681 ± 3.6762 | 0.2994 ± 0.1827 |
| Progesterone | | | |
| Soft gel Capsule | | | |
| 200 mg, | | | |
| (Single dose) | | | |
| Oral | | | |
| Reference | 159.2795 ± 204.2120 | 3.4829 ± 3.0843 | 0.3209 ± 0.1906 |
| product | | | |
| PROMETRIUM ® | 114.6482 ± 137.7684 | 3.4296 ± 2.9995 | 0.3485 ± 0.2491 |
| (Progesterone) | | | |
| soft gel Capsule | | | |
| 200 mg | | | |
| (Single dose - 2 × | | | |
| 200 mg), | | | |
| Oral | | | |

Bioequivalence Analysis

In this study, the within-subject standard deviation of the reference formulation (S_{WR}) was found to be ≧0.294 for Cₘₐₓ and AUC (AUC₀₋ₜ and AUC_{0-∞}). As a result, the point estimate (test/reference geometric mean ratio) and 95% upper confidence bound for (μ_{T}−μ_{R})²−(θS²_{WR}) was determined using ln-transformed data using SAS® statistical software version 9.2 from SAS Institute Inc, USA. This methodology (Scaled-Average Bioequivalence (“SABE”)) is consistent with FDA guidelines for calculating bioequivalence for highly variable drugs, such as progesterone. Using the SABE methodology, T demonstrated improved bioavailability compared to PROMETRIUM and was considered superior to PROMETRIUM. Supporting data is shown in Tables 12 and 13 below.

TABLE 12
Point of estimate, Within-subject SD (S_{wr}) and 95% Upper
Confidence Bound of Test product (T) versus Reference
product (R) for, Progesterone (Baseline corrected)

| | Point | | Upper 95% |
|---|---|---|---|
| | Estimate | Within-Subject | Confidence |
| Parameter | (T/R ratio) | SD (S_{wr}) | Bound |
| Cₘₐₓ | 1.38 | 1.1334 | −0.481956 |
| (ng/mL) | | | |
| AUC₀₋ₜ | 1.28 | 0.8908 | −0.326613 |
| (ng · hr/mL) | | | |
| AUC_{0-∞} | 1.28 | 0.7704 | −0.135158 |
| (ng · hr/mL) | | | |

TABLE 13
Point of estimate, Within-subject SD (Swr) and 95% Upper
Confidence Bound of Test product (T) versus Reference
product (R) for, Progesterone (Baseline Uncorrected)

| | Point of | | 95% Upper |
|---|---|---|---|
| | estimate | Within-subject | Confidence |
| Parameter | (T/R ratio) | SD (S_{wr}) | Bound |
| Cₘₐₓ | 1.38 | 1.1333729 | −0.481836 |
| (ng/mL) | | | |
| AUC₀₋ₜ | 1.28 | 0.8907574 | −0.326277 |
| (ng · hr/mL) | | | |
| AUC_{0-∞} | 1.29 | 0.7704431 | −0.134134 |
| (ng · hr/mL) | | | |

In view of the data noted above, the appropriate dosage of progesterone in the formulation disclosed herein necessary to achieve bioequivalence to PROMETRIUM was 150 mg. The computed results are shown in Table 14. This suggests that, in certain embodiments, the formulations disclosed herein have nearly 25% greater bioavailability than the current marketed formulation (PROMETRIUM).

TABLE 14
Summary of Evaluations of Baseline-Corrected Progesterone
Results for a computed 150 mg Test Capsule vs. a 200
mg PROMETRIUM ® Capsule

| | Point | Within-Subject | Upper 95% |
|---|---|---|---|
| Parameter | Estimat | SD (S_{wr}) | Confidence |
| Cₘₐₓ | 1.03 | 1.1334 | −0.746836 |
| (ng/mL) | | | |
| AUC₀₋ₜ | 0.96 | 0.8908 | −0.465204 |
| (ng · hr/mL) | | | |

### Example 16

### Bioavailability Assessment—Fed #3

The amounts progesterone administered include 225 mg/day and 300 mg/day of progesterone. Progesterone capsule sizes are 75 mg and 150 mg capsules. Subjects taking the progesterone capsules are compared to subjects taking placebos. In both cases subjects are estrogen-primed.

The study includes: approximately a 6-week (42 days) screening period before enrolling into the study; approximately 6 weeks of Estrace®-priming before randomization; 6 weeks of blinded treatment (along with Estrace® treatment); and up to approximately 5 weeks of follow-up. The study is a phase 3, randomized, three-cycle, double-blind, placebo-controlled study to evaluate induction of secretory conversion of endometrium and withdrawal bleeding after administration of progesterone in estrogen-primed women with secondary amenorrhea. In clinical facilities, at the first visit (baseline-Cycle 1, day 1) subjects are estrogen-primed using an oral estradiol (i.e. 1.0 mg Estrace®). This priming takes place for 25 days. Compliance with estrogen-priming is determined (throughout, and at day 28−3 day to +1 day). Subjects will begin cycle 2 of estrogen-priming (Cycle 2, day 1).

After 12 days (±2 days), subjects return to clinic. A transvaginal ultrasound (TVU) is conducted. Estrogen compliant subjects, and subjects meeting other criteria (i.e. double-walled endometrial thickness of ≧5 mm, 280% compliant with Estrace, and negative urine pregnancy test) are randomized for treatment with progesterone.

Subjects begin blinded administration on day 14 of Cycle 2. Subjects continue both Estrace® and blinded administration through day 25 of Cycle 2. No medication is taken from Cycle 2, Day 26-28.

Estrace® 1.0 mg is re-started at Cycle 3, Day 1 and continued until Day 25. Subjects will return to the clinic at Cycle 2, Day 12 (±2d) for study assessments. At Cycle 3, Day 14, subjects will again begin taking blinded study medication through Day 25.

Subjects return to the clinic day 24 (±1 day) of Cycle 3, at which time an endometrial biopsy is conducted.

Subjects complete their final dose of Estrace® and blinded study medication on Day 25 and return to the clinic for a follow-up visit approximately 10 days later (upon receipt of biopsy results). Final visit assessments are conducted. Subjects whose endometrial biopsy results show proliferative endometrium are prescribed a 14 day course of medroxyprogesterone acetate 10 mg [MPA] as standard-of-care treatment to counterbalance the effect of estrogen-induced endometrial proliferation. These subjects receive a follow up telephone call at 2-4 weeks after completion of the MPA course and queried for the incidence of bleeding and adverse events. Unscheduled visits are allowed as needed.

### Example 17

An open-label, balanced, randomized, two-treatment, two-period, two-sequence, single-dose, crossover, oral bioequivalence study was conducted with progesterone soft gel capsules having the formulation disclosed in Table 9 as fill material and PROMETRIUM® soft gel capsule 200 mg in normal healthy, adult human male subjects under fasting conditions.

A total of 25 normal healthy, adult, human male subjects were enrolled into the study. All subjects were housed in the clinical facility for at least 11 hours before dosing through a 24 hours post dose. After an overnight fast of at least 10 hours, a single dose of either test product (T) or reference product (R) (as per a randomization schedule) was administered orally to each subject with 240 mL of water. There was a washout period of 14 days between treatments. 18 blood samples were collected at: −1 hours, −0.5 hours, 0 hours, 0.25 hours, 0.5 hours, 0.67 hours, 0.83 hours, 1.00 hours, 1.33 hours, 1.67 hours, 2.00 hours, 2.50 hours, 3.00 hours, 4.00 hours, 6.00 hours, 8.00 hours, 12.00 hours, and 24.00 hours. The testing indicated that T and R had the following PK parameters:

TABLE 15
Summary of Primary Pharmacokinetic Profile of Test product (T),
Progesterone soft gel Capsule 200 mg (Baseline Corrected)

| Pharmacokinetic | Geometric | Arithmetic | Standard |
|---|---|---|---|
| Parameter | Mean* | Mean | Deviation |
| Cₘₐₓ (ng/mL) | 0.9701 | 1.1767 | 1.7458 |
| AUC₀₋ₜ (ng · hr/mL) | 2.4130 | 4.5380 | 8.2350 |
| AUC_{0-∞} (ng · hr/mL) | 27.2091 | 36.9118 | 27.8580 |

*Estimate of Least Square Mean used to calculate Geometric Mean

TABLE 16
Summary of Primary Pharmacokinetic Profile of Reference
product (R), PROMETRIUM ® (Progesterone)
soft gel Capsule 200 mg (Baseline Corrected)

| Pharmacokinetic | Geometric | Arithmetic | Standard |
|---|---|---|---|
| Parameter | Mean* | Mean | Deviation |
| Cₘₐₓ (ng/mL) | 2.0929 | 2.9877 | 3.1620 |
| AUC₀₋ₜ (ng · hr/mL) | 4.9870 | 7.6108 | 7.0148 |
| AUC_{0-∞} (ng · hr/mL) | 13.1050 | 26.8905 | 55.3784 |

*Estimate of Least Square Mean used to calculate Geometric Mean

TABLE 17
T/R Ratio and 90% Confidence Intervals of Test product (T) versus
Reference product (R) for, Progesterone (Baseline Corrected)

| Pharmacokinetic | | 90% Confidence |
|---|---|---|
| Parameter | T/R Ratio % | Intervals |
| Cₘₐₓ (ng/mL) | 46.35% | 34.3% to 62.63% |
| AUC₀₋ₜ (ng · hr/mL) | 48.39% | 25.84% to 90.62% |
| AUC_{0-∞} (ng · hr/mL) | 207.62% | 72.18% to 597.25% |

This data indicates that T and R are not bioequivalent because the 90% confidence interval of the least square mean of Cₘₐₓ, AUC₀₋ₜ and AUC_{0-∞} were 34.3% to 62.63%, 25.84% to 90.62%, and 72.18% to 597.25% respectively. They were thus not within the limit of 80.00% and 125.00% used by the FDA to demonstrate bioequivalence.

### Example 18

An open-label, balanced, randomized, two-treatment, two-period, two-sequence, single-dose, crossover, oral bioequivalence study was conducted with progesterone soft gel capsules having the formulation disclosed in Table 9 as fill material and PROMETRIUM® soft gel capsule 200 mg in normal healthy, adult human male subjects under fed conditions.

A total of 25 normal healthy, adult, human male subjects were enrolled into the study. All subjects were housed in the clinical facility for at least 11 hours before dosing through a 24 hours post dose. After an overnight fast of at least 10 hours, a high fat, high calorie breakfast was served 30 minutes before administering a single dose of either test product (T) or reference product (R) (as per a randomization schedule). Capsules were given to each subject orally with 240 mL of water. There was a washout period of 14 days between treatments. 18 blood samples were collected at: −1 hours, −0.5 hours, 0 hours, 0.25 hours, 0.5 hours, 0.67 hours, 0.83 hours, 1.00 hours, 1.33 hours, 1.67 hours, 2.00 hours, 2.50 hours, 3.00 hours, 4.00 hours, 6.00 hours, 8.00 hours, 12.00 hours, and 24.00 hours. The testing indicated that T and R had the following PK parameters:

TABLE 18
Summary of Primary Pharmacokinetic Profile of Test product (T),
Progesterone soft gel Capsule 200 mg (Baseline Corrected)

| Pharmacokinetic | Geometric | Arithmetic | Standard |
|---|---|---|---|
| Parameter | Mean* | Mean | Deviation |
| Cₘₐₓ (ng/mL) | 20.8344 | 88.1233 | 165.6133 |
| AUC₀₋ₜ (ng · hr/mL) | 42.6781 | 124.7467 | 215.4315 |
| AUC_{0-∞} (ng · hr/mL) | 59.0419 | 150.9140 | 237.6730 |

*Estimate of Least Square Mean used to calculate Geometric Mean

TABLE 19
Summary of Primary Pharmacokinetic Profile of Reference
product (R), PROMETRIUM ® (Progesterone)
soft gel Capsule 200 mg (Baseline Corrected)

| Pharmacokinetic | Geometric | Arithmetic | Standard |
|---|---|---|---|
| Parameter | Mean* | Mean | Deviation |
| Cₘₐₓ (ng/mL) | 12.4661 | 41.5344 | 87.8350 |
| AUC₀₋ₜ (ng · hr/mL) | 29.9365 | 60.0080 | 105.0084 |
| AUC_{0-∞} (ng · hr/mL) | 36.9906 | 65.4258 | 109.0883 |

*Estimate of Least Square Mean used to calculate Geometric Mean

TABLE 20
T/R Ratio and 90% Confidence Intervals of Test product (T) versus
Reference product (R) for, Progesterone (Baseline Corrected)

| Pharmacokinetic | | 90% Confidence |
|---|---|---|
| Parameter | T/R Ratio % | Intervals |
| Cₘₐₓ (ng/mL) | 167.13% | 79.38% to 351.89% |
| AUC₀₋ₜ (ng · hr/mL) | 142.56% | 85.01% to 239.08% |
| AUC_{0-∞} (ng · hr/mL) | 159.61% | 103.59% to 245.94% |

This data indicates that T and R are not bioequivalent because the 90% confidence interval of the least square mean of Cₘₐₓ, AUC₀₋ₜ, and AUC_{0-∞} were 79.38% to 351.89%, 85.01% to 239.08%, and 103.59% to 245.94%. They were thus not within the limit of 80.00% and 125.00% used by the FDA to demonstrate bioequivalence. But importantly, and unlike the fasted study, the fed study demonstrated that test product T demonstrated enhanced oral bioavailability vs. PROMETRIUM®.

It will be apparent to those skilled in the art that various modifications and variations can be made in this disclosure without departing from the spirit or scope of the disclosure. Thus, it is intended that this disclosure cover the modifications and variations of this disclosure provided they come within the scope of the appended claims and their equivalents.

Likewise, numerous characteristics and advantages have been set forth in the preceding description, including various alternatives together with details of the structure and function of the devices or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

What is claimed is:

1. A pharmaceutical composition for administering progesterone to subject in need thereof, the composition comprising an amount of progesterone and one or more solubilizing agents, wherein upon administration to the subject in need thereof, the composition produces at least one the following pharmacokinetic parameters in said subject:
a. an AUC₀₋ₜ in (ng/ml)*hr of from about 5 to about 500;
b. an AUC_{0-∞} in (ng/ml)*hr of from about 5 to about 500; or
c. a Cₘₐₓ in ng/ml of from about 3 to about 350;
wherein the amount of progesterone is less than 200 mg.

2. The pharmaceutical composition of claim 1, wherein the AUC₀₋ₜ is from about 5 to about 400, from about 5 to about 300, from about 5 to about 240, from about 20 to about 200, from about 25 to about 150, or from about 25 to about 140.

3. The pharmaceutical composition of claim 1, wherein the AUC₀₋ₜ is 120 (ng/ml)*hr±95%.

4. The pharmaceutical composition of claim 1, wherein the AUC_{0-∞} is from about 5 to about 400, from about 5 to about 300, from about 5 to about 270, from about 20 to about 200, from about 25 to about 150, or from about 25 to about 140.

5. The pharmaceutical composition of claim 1, wherein the AUC_{0-∞} is 137 (ng/ml)*hr+95%.

6. The pharmaceutical composition of claim 1, wherein the Cₘₐₓ in ng/ml is from about 3 to about 325, from about 3 to about 300, from about 3 to about 250, from about 3 to about 240, or from about 3 to about 230.

7. The pharmaceutical composition of claim 1, wherein the Cₘₐₓ is 75 ng/ml±95%.

8. The pharmaceutical composition of claim 1, wherein the amount of progesterone is about 150 mg.

9. The pharmaceutical composition of claim 8, wherein the amount of progesterone is 150 mg.
